(19) United States
(12) Patent Application Publication
(10) Pub. No.: US 2023/0172968 A1
(43) Pub. Date: Jun. 8, 2023

(54) CONTROLLED RELEASE HYALURONIC ACID COMPOSITIONS

(71) Applicant: Prohibix LLC, Philadelphia, PA (US)

(72) Inventor: Brendan Patrick PURCELL, Philadelphia, PA (US)

(21) Appl. No.: 17/919,410

(22) PCT Filed: Apr. 19, 2021
(86) PCT No.: PCT/US21/27912

§ 371 Date: Oct. 17, 2022

Related U.S. Application Data
(60) Provisional application No. 63/011,996, filed on Apr. 17, 2020.
(60) Provisional application No. 63/136,508, filed on Jan. 12, 2021.

(51) Int. Cl. A61K 31/728; A61K 9/00; A61K 45/06

(52) U.S. Cl. CPC A61K 31/728; A61K 9/0034; A61K 9/0019; A61K 45/06

(57) ABSTRACT

The disclosure concerns high molecular weight hyaluronic acid compositions that release hyaluronic acid at controlled rates. The compositions may achieve improved retention and duration of hyaluronic acid in the body, and may be used in methods of treating tissues throughout the body. Compositions, methods of treating, and methods of making are described.

### CROSS REFERENCE TO RELATED APPLICATIONS

This application claims the benefit of U.S. provisional application No. 63/011,996, which was filed Apr. 17, 2020 and titled HYALURONIC ACID PARTICLE COMPOSITIONS TO MODULATE VICOELASTIC PROPERTIES, and U.S. provisional application No. 63/136,508, which was filed Jan. 12, 2021 and titled SUSTAINED RELEASE HYALURONIC ACID COMPOSITIONS, both of which are incorporated by reference as if fully set forth.

### FIELD

The disclosure relates to particles composed of hyaluronic acid for local administration to the body.

### BACKGROUND

Hyaluronic acid (HA) is a linear glycosaminoglycan composed of repeating D-glucuronic acid and N-acetyl-D-glucosamine disaccharides. It is a hydrophilic, water-swollen polymer, and is abundant in the extracellular matrix of tissues throughout the body. HA has important mechanical and biological roles in regulating tissue hydration and mechanics, tissue formation, wound healing, and inflammation. HA polymers can be found in the body at molecular weights ranging from 5,000 daltons (Da) to 20,000,000 Da. HA dissolved in aqueous buffers form shear-thinning solutions with viscoelastic properties that are directly proportional to HA concentration and molecular weight [1]. HA is present in the normal synovial fluid of joints at approximately 2 million Da and at a concentration of 2-4 mg/mL to produce a clear viscous liquid [2-4]. In an aged or diseased joint, the concentration of higher molecular weight HA is lower [5]. This has implications on the mechanical and lubricating properties of the synovial fluid as well as the dysregulation of biological processes that results in persistent inflammation and synovitis in the joint [6]. Similarly, the HA content in the vitreous of the eye has been shown to decrease with age, altering viscoelastic properties of the vitreous which is thought to ultimately impair eye function [7], and HA degradation fragments have been shown to cause inflammation during intervertebral disc degeneration [8]. Studies have shown that HA molecular weights in the range of 500 kDa to 4 MDa exhibit the greatest ability to reduce inflammation and stimulate production of endogenous HA [9, 10].

The synovial fluid is contained by a synovial membrane that is an elastic tissue composed of fibrous collagen and synovial tissue cells. The synovial membrane interstitium is microporous and transports macromolecules including HA from the synovial fluid to the blood plasma [11]. The turnover of HA in the synovial fluid is slower than other smaller molecules such as albumin but is still less than 24 hrs [12]. Increasing the molecular weight of exogenous HA from 100 kDa to 6 MDa only increases the half-life from 10 hrs to 13 hrs [12]. Therefore, it is challenging to sustain exogenous HA in the synovial fluid following IA injections in order to maintain synovial fluid viscoelastic properties for therapeutically relevant times.

Table 1 shows HA molecular weight, concentration and viscoelastic properties of human synovial fluid and HA viscosupplement products. Data were compiled from published literature [2-5, 13-15].

TABLE 1

| | Molecular | | Zero Shear | Storage | Loss |
|---|---|---|---|---|---|
| | Weight | Concentration | Viscosity | Modulus | Modulus |
| | (kDa) | (mg/mL) | (Pa sec) | (G′, Pa) | (G″, Pa) |
| Healthy | 2,000 | 2.5 to 3.8 | 1 to 175 | ~12 @ 0.5 Hz | ~8.6 @ 0.5 Hz |
| Synovial Fluid | | | | ~26 @ 2.5 Hz | ~3.8 @ 2.5 Hz |
| Osteoarthritis | 1,900 | 1.55 ± | 3.40 ± 2.90; | 2.14 ± 1.7/~0.32 @ 0.5 Hz | 1.63 ± 1/~0.48 @ 0.5 Hz |
| Synovial Fluid | | 0.14/1.3 ± 0.5 | 0.1 to 1 | 3.55 ± 2.72/~1.8 @ 2.5 Hz | 2.51 ± 1.25/~1.9 @ 2.5 Hz |
| Hyalgan | 500 to | 20 | 0.27 | 0.03 @ 0.5 Hz | 0.69 @ 0.5 Hz |
| | 730 | | | | |
| Supartz | 620 to | 10 | 3.07 | 2.2 @ 0.5 Hz | 6.5 @ 0.5 Hz |
| | 1170 | | | 11.8 @ 2.5 Hz | 18.1 @ 2.5 Hz |
| Orthovisc | 1,000 to | 15 | 120.8 | 51.2/79.5 @ 0.5 Hz | 45.9/41.5 @ 0.5 Hz |
| | 2,900 | | | 111.2/147.4 @ 2.5 Hz | 61.5/68.2 @ 2.5 Hz |
| Eufllexa | 2,400 to | 20 | 91.2 | 57.4 @ 0.5 Hz | 32.1 @ 0.5 Hz |
| | 3,600 | | | 93.2 @ 2.5 Hz | 34.8 @ 2.5 Hz |
| Synvisc | 1,000 to | 8 | 191.7 | 91.9/~60/79.9 @ 0.5 Hz | 26.1/~23/20.3 @ 0.5 Hz |
| (Hylan A) | 2,000 | | | 118.1/~85/99.2 @ 2.5 Hz | 22.5/~21/17.8 @ 2.5 Hz |
| Durolane | 1,000 | 20 | | ~480 @ 0.5 Hz | ~68 Pa @ 0.5 Hz |
| | | | | ~580 @ 2.5 Hz | ~68 Pa @ 2.5 Hz |
| GelOne | 900 | 10 | 190.2 | 11.0 @ 0.5 Hz | 3.2 @ 0.5 Hz |
| | | | | 15.0 @ 2.5 Hz | 5.9 @ 2.5 Hz |
| [symbol] | | | | [symbol] | [symbol] |
| | | | | [symbol] | [symbol] |

Recognizing the decreased HA content and molecular weight in tissues throughout the body, exogenous solutions of HA are routinely administered through local injection procedures to supplement lost or degraded HA in a tissue or joint. While exogenous administration of uncrosslinked, high molecular weight HA has been utilized to maintain the viscoelastic and biologic functions of HA (Hyalgan, Orthovisc), the retention and duration of uncrosslinked HA is relatively short (half-life less than 24 hrs) [12]. To enhance retention and duration of exogenously administered HA, chemical crosslinking of HA has been utilized. HA is most commonly crosslinked with 1,4-Butanediol diglycidyl ether (BDDE) through a condensation reaction under elevated temperature and basic pH. A stable ether bond is formed between a hydroxyl group on HA and the epoxide group on BDDE. By controlling the extent of BDDE crosslinking, viscous HA solutions and hydrogels can be fabricated with a range of physical properties. For example, a range of BDDE crosslinked HA dermal filler formulations have been developed with a different swelling, stiffness and tissue distribution characteristics [16]. In addition, HA derived from rooster combs which contains residual proteins has been crosslinked with glutaraldehyde to different extents for a range of physical properties and durations in the body [17, 18].

Crosslinks can be introduced at low extents of crosslinking (typically a few percent of the repeat HA disaccharide) between linear HA polymers to effectively increase the molecular weight of the HA which remains soluble in water, or at higher extents of crosslinking (typically five percent or greater) to form connected networks that are insoluble, water-swollen hydrogels.

HA formulations with more than one HA crosslink density have been developed for improved HA stability. For example, Synvisc (Sanofi Genzyme) is comprised of hylan B particles (high divinyl sulfone (DVS) crosslink density) swollen within a solution of hylan A (HA crosslinked to increase molecular weight) (U.S. Pat. No. 5,143,724). Hylan B has a longer synovial half-life compared to hylan A [17], and hylan B particles have been recovered from the synovial fluid of goats 28 days after intra-articular injection [18], however the BDDE crosslinked particles remain a solid gel covered with immune cells indicating phagocytosis so it is unclear what activity the crosslinked HA particle have in the synovial fluid. Similarly, UtopyHA (Luminera) comprises highly crosslinked HA particles mixed with crosslinked HA and uncrosslinked HA to form a homogenous formulation with no detectable phase separation or boundaries for enhanced formulation stability (WO2020194294A1).

Degradation of crosslinked HA formulations is regulated by both the breakdown of the HA polymer chain through enzyme activity or oxidative cleavage of glycosidic bonds, and the breakdown of bonds in the crosslinker [19]. The ether bonds formed during BDDE and DVS crosslinking of HA, as well as the ether bonds along the backbone of BDDE are relatively stable in water allowing for shelf-stability of water swollen formulations for up to two years at room temperature as well as persistence in the dermis for a year or more [19]. Therefore, the degradation of BDDE crosslinked HA products in the body is through breakdown of the HA backbone and not the crosslink and minimal HA is released.

Catabolic hyaluronidase enzymes are upregulated in inflamed tissues including arthritic joints leading to an increased turnover of HA. Several molecules have been shown to directly inhibit hyaluronidase activity including sulfated polysaccharides such as heparin [20, 21]. In addition, chemical modification of HA, including the introduction of sulfate groups, has been shown to prevent hyaluronidase degradation of the modified HA [22].

While the majority of commercial HA viscosupplement products rely of crosslinking with 1,4-Butanediol diglycidyl ether (BDDE), a variety of chemically modified HAs have been developed for enhanced control of material properties [23]. HA can be modified with reactive groups through chemical conjugation to the hydroxyl or carboxylic acid groups along the HA backbone. Upon mixing with initiator chemistries or crosslinkers with complimentary reactive groups, liquid solutions of HA can be transformed into solid, water-swollen hydrogels. The crosslinking can be either through covalent bond formation or non-covalent interactions such as electrostatic, hydrogen bonding, hydrophobic and van der Walls forces. HAs of relatively low molecular weight (less than 200 kDa) are commonly used to produce precursor solutions with low viscosities to allow for initiators and crosslinkers to be uniformly mixed for the formation of hydrogels with controlled physical properties.

Crosslinking chemistries that are degradable have been incorporated into HA hydrogels towards the fabrication of gels with controlled degradation. For example, US20120114615A1 describes hydrolytically liable ester groups disposed between a reactive methacrylate and the HA backbone through chemical conjugation of lactic acid and hydroxyethyl methacrylate derivatives. In addition, U.S. Pat. No. 9,694,081 describes protease degradable peptides that are disposed between reactive crosslinking groups and the HA backbone for controlled hydrogel degradation in the presence of protease activity.

### SUMMARY

In an aspect, the invention relates to a composition comprising: a first fraction of hyaluronic acid and a second fraction of hyaluronic acid. When in an aqueous medium, the first fraction and the second fraction are insoluble and the composition releases the hyaluronic acid from the first fraction to the aqueous medium at a rate different than from the second fraction.

In an aspect, the invention relates to a composition comprising: a first fraction of hyaluronic acid and a second fraction of hyaluronic acid. The second fraction is encapsulated in the first fraction. When in an aqueous medium, the first fraction and the second fraction are insoluble and the composition releases the hyaluronic acid from the first fraction to the aqueous medium at a rate different than from the second fraction.

In an aspect, the invention relates to a method of treatment. The method comprises administering a composition to a subject in need thereof. The composition comprises a first fraction of hyaluronic acid and a second fraction of hyaluronic acid. The second fraction is encapsulated in the first fraction. When in an aqueous medium, the first fraction and the second fraction are insoluble and the composition releases the hyaluronic acid from the first fraction to the aqueous medium at a rate different than from the second fraction.

In an aspect, the invention relates to a method of making a composition. The method comprises encapsulating a second fraction in a first fraction of hyaluronic acid. The first fraction limits swelling and therefore release of the second fraction.

In an aspect, the invention relates to a method of administering hyaluronic acid. The method comprises injecting a composition comprising two insoluble fractions of hyaluronic acid that release hyaluronic acid at different rates into a body through a syringe or catheter.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 shows the proposed viscoelastic effects of HA release from a composition comprising two HA particle fractions in the synovial fluid. Current HA formulations have enhanced viscoelastic effects that are short lived in the synovial fluid as HA is cleared. By releasing HA from insoluble fractions at different rates, multiple phases of increased viscoelasticity can be combined for sustained viscoelastic effects.

FIGS. 2A and 2B show a HA hydrogel schematic comprising two fractions of HA: uncrosslinked HA particles encapsulated within a HA hydrogel crosslinked with hydrolytically degradable chemistries at a crosslink density of 10.4 mM (HAX MED) (A). The hydrogels were incubated in physiological buffer and the surrounding fluid was collected and replaced every day and HA content was quantified with a carbazole assay (B, n=3 gels per group, mean+/−standard deviation). HA content is presented per milliliter of starting hydrogel.

FIGS. 3A and 3B shows a HA hydrogel schematic comprising three fractions of HA: uncrosslinked HA particles (HAP) and crosslinked HA particles with a crosslink density of 6.7 mM (HAX LO) before being fragmented into microparticles, dehydrated and encapsulated in a HA hydrogel with a crosslink density of 22 mM (HAX HI) (A). The crosslinks in both crosslinked phases are hydrolytically degradable. The hydrogels were incubated in physiological buffer and the surrounding fluid was collected and replaced every day and HA content was quantified with a carbazole assay (B, n=3 gels per group, mean+/−standard deviation). HA content is presented per milliliter of starting hydrogel. A HAX LO hydrogel (20 mg/mL HA concentration) was incubated physiological buffer and HA release quantified for comparison to encapsulated HAX LO particles (inset, n=3 gels per group, mean+/−standard deviation).

FIG. 4 shows the swelling ratio of HA hydrogels with encapsulated crosslinked and uncrosslinked fractions (n=3 gels per group, mean+/−standard deviation).

FIGS. 5A and 5B shows a HA hydrogel schematic comprising three fractions of HA: uncrosslinked HA particles (HAP) and HA hydrogels crosslinked with a crosslink density of 22 mM (HAX HI) before being dehydrated and encapsulated in a second HA hydrogel with a crosslink density of 11 mM (HAX MED) (FIG. 5A). The crosslinks in both crosslinked phases are hydrolytically degradable. The hydrogels were incubated in physiological buffer and the surrounding fluid was collected and replaced every day and HA content was quantified with a carbazole assay (FIG. 5B, n=3 gels per group, mean+/−standard deviation). HA content is presented per milliliter of starting hydrogel.

FIGS. 6A and 6B shows a schematic of different phases of HA release that correspond to the different fractions of HA in the three-fraction hydrogel microparticles (A). In phase I, uncrosslinked HA is released at a high concentration to increase the viscosity of the surround fluid. In phase II, crosslinked HA is released and the particles swell to increase viscoelastic properties. In phase III, the crosslinked HA particles are partially degraded resulting in swelling and release of crosslinked HA. The viscoelastic properties of the three fraction microparticles were measured after different incubation times (37 degrees C., 60 rotations per minute, PBS, supernatant buffer changed daily). Phase I corresponds to day 2, Phase II corresponds to day 15 and Phase III corresponds to day 30. Loss and storage modulus were measured at 0.5% strain and 2.5 Hz. Viscosity values were measured over 1E-3 to 1E3 1/s shear rates. All measurements at 22 degrees C. with a 20 mm plate geometry (TA HR20 rheometer). *p<0.05 compared to PBS control, student's t-test.

FIGS. 7A and 7B shows the mass fraction of HA released from three fraction HA microparticles into phosphate buffered saline at 37° C., 80 rpm. The fractions were 4 wt % uncrosslinked HA particles and 11 wt % crosslinked HA particles (6.1 mM crosslink density) encapsulated in a 5 wt % HA hydrogel (23 mM crosslink density) that was then processed into microparticles. All crosslinks between HA polymers contained hydrolytically degradable ester groups. Data shown are mean+/−standard deviation, n=3 samples.

FIGS. 8A and 8B shows the mass fraction of HA released from three fraction HA microparticles into phosphate buffered saline at 37° C. 80 rpm. The fractions were 8.5 wt % crosslinked HA particles (2.7 mM crosslink density) and 5.5 wt % crosslinked HA particles (7.9 mM crosslink density) encapsulated in a 6 wt % HA hydrogel (27 mM crosslink density) that was then processed into microparticles. All crosslinks between HA polymers contained hydrolytically degradable ester groups. Data shown are mean+/−standard deviation, n=3 samples.

FIGS. 9A and 9B shows the mass fraction of HA released from three fraction HA microparticles into phosphate buffered saline at 37° C., 80 rpm. The fractions were 5 wt % uncrosslinked HA particles and 10 wt % crosslinked HA particles (4.1 mM crosslink density) encapsulated in a 5 wt % HA hydrogel (16 mM crosslink density) that was then processed into microparticles. All crosslinks between HA polymers contained hydrolytically degradable ester groups. Data shown are mean+/−standard deviation, n=3 samples.

FIGS. 10A and 10B shows the mass fraction of HA released from four fraction HA microparticles into phosphate buffered saline at 37° C., 80 rpm. The fractions were 4 wt % uncrosslinked HA particles, 4 wt % crosslinked HA particles (3.3 mM crosslink density) and 7 wt % crosslinked HA particles (9.9 mM crosslink density) encapsulated in a 5 wt % HA hydrogel (28 mM crosslink density) that was then processed into microparticles. All crosslinks between HA polymers contained hydrolytically degradable ester groups. Data shown are mean+/−standard deviation, n=3 samples.

### DETAILED DESCRIPTION

Certain terminology is used in the following description for convenience only and is not limiting. The words “right,” “left,” “top,” and “bottom” designate directions in the drawings to which reference is made.

“Hydrogel” is any network of crosslinked hydrophilic polymers wherein the storage modulus (G′) is greater than the loss modulus (G″). Hydrogels are considered insoluble and can have water contents from 10 to 99.9% w/w.

“Crosslink” refers to covalent or non-covalent bonds formed between polymers.

“Crosslinker” refers to molecules that form bonds between polymers. Crosslinkers can react with polymer to form a crosslinking bond or be chemically modified onto the polymer prior to crosslinking to other polymers.

“Hydrolytically degradable” refers to a crosslink that contains covalent bonds that dissociate due to nucleophilic attack of a water molecule at a rate faster than the dissociation of an ether bond in water.

“Fraction” is a subset of HA polymers that can be identified by differences in molecular weight, extent or type of chemical crosslinking, extent or type of chemical modification or crosslink density. These subsets may have a distribution around these properties but can be identified through a non-zero difference in the means of their distributions. These subsets are produced separately through different chemical synthesis or formulation process steps.

“Extent of chemical modification” refers to the percent of repeat disaccharides on an HA polymer that are modified with a new chemical group. For HA, the repeat unit is the disaccharide composed of D-glucuronic acid and N-acetyl-D-glucosamine, linked via alternating β-(1→4) and β-(1→3) glycosidic bonds.

“Extent of chemical crosslinking” refers to the percent of repeat disaccharides on an HA polymer that are bound to another HA polymer or linker molecule through covalent or non-covalent interactions. For HA, the repeat unit is the disaccharide composed of D-glucuronic acid and N-acetyl-D-glucosamine, linked via alternating β-(1→4) and β-(1→3) glycosidic bonds.

“Crosslink density” refers to the concentration of bonds between crosslinker and polymer per unit volume. Crosslink density can be altered through changes in polymer concentration in addition to extents of chemical modification or crosslinking. When used to describe crosslinked HA networks, it is the concentration of bonds to HA per unit volume.

“Drug” is any chemical substance of known structure that when administered to a living organism, produces a biological effect.

“Medicament” is any chemical substance used for medical treatment.

“Molecular weight” is a measure of the size of a polymer. As used herein, the term “molecular weight” refers to the number average molecular weight of a polymer as determined by gel permeation chromatography in aqueous buffer.

“Functionality” refers to the number of chemical groups per molecule that are capable of crosslinking to another molecule.

“Storage modulus” or (G′) is a measure of stored energy under force and represents the elastic portion of the complex modulus of viscoelastic materials. G′ can be determined by oscillatory rheology.

“Loss modulus” or (G″) is a measure of energy dissipation under force and represents the viscous portion of the complex modulus of viscoelastic materials. G″ can be determined by oscillatory rheology.

“Zero shear viscosity” is the viscosity measured at a shear rate approaching zero and is determined by fitting experimental data to mathematical models.

“Liquid” refers to an aqueous solution wherein the storage modulus (G′) is less than or equal to the loss modulus (G″).

“Released” refers to separation of soluble HA from insoluble HA.

“Polymer mass fraction” is the percent of the total polymer in the composition. This can refer to the fraction of polymer that is released from a composition over a specific amount of time.

“Encapsulated” refers to being partially or completely surrounded by and within an insoluble, crosslinked fraction. Encapsulated fractions cannot be physically separated without breaking crosslinks or polymer chains of the encapsulating fractions. Encapsulated fractions and encapsulated drugs can diffuse out of encapsulating fractions.

“wt %” is used to describe the mass in grams of dry material powder added per milliliter of buffer during composition fabrication. For example, adding 0.1 gram of powder to 1 milliliter of buffer is 10 wt %.

“Insoluble” refers to having structural integrity to not dissolve into a uniform solution when placed in buffer within 24 hours at room temperature

“Soluble” refers to dissolving into a uniform solution when placed in buffer within 24 hours at room temperature.

“Physiological conditions” refers to pH 7.4 and 37 degrees Celsius.

It is also to be understood that the terminology used herein is for the purpose of describing particular aspects only and is not intended to be limiting. As used in the specification and in the claims, the term “comprising” is open ended and does not restrict the addition of other elements. But “comprising” encompasses embodiments of “consisting of” and “consisting essentially of” as interpreted in the United States. Unless defined otherwise, all technical and scientific terms used herein have the same meaning as commonly understood by one of ordinary skill in the art to which this disclosure belongs.

As used in the specification and the appended claims, the singular forms “a,” “an” and “the” include plural referents unless the context clearly dictates otherwise. Thus, for example, reference to “a crosslinking polymer” includes mixtures of two or more crosslinking polymers. In addition, reference to “a pharmaceutical carrier” includes mixtures of two or more such carriers, and the like.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed. Every range herein may be divided into subranges. A subrange may have a lower endpoint selected from any value less than the highest value of the range. The lower endpoint may be selected by choosing the lowest endpoint of the range or any incremental value, incremented by “one” in the lowest decimal place expressed in the original range, excluding the highest value in the range. A subrange may have a higher endpoint selected from any value higher than the lowest value of the range. The high endpoint may be selected by choosing the any incremental value in the range, incremented by “one” in the lowest decimal place expressed in the original range, excluding the lowest value in the range.

As used herein, the terms “about” and “at or about” mean that the amount or value in question can be the designated value, approximately the designated value, or about the same as the designated value. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise. Any particular number herein may be modified with the term “exact” or “exactly” to form an additional embodiment herein.

As used herein, the terms “optional” or “optionally” means that the subsequently described event or circumstance may or may not occur, and that the description includes instances where said event or circumstance occurs and instances where it does not. For example, the phrase “optionally substituted alkyl” means that the alkyl group may or may not be substituted and that the description includes both substituted and unsubstituted alkyl groups.

The phrase “at least one” followed by a list of two or more items, such as “A, B, or C,” means any individual one of A, B or C as well as any combination thereof.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary.

Each of the materials disclosed herein are either commercially available and/or the methods for the production thereof are known to those of skill in the art.

An embodiment includes a composition comprising a first fraction of hyaluronic acid and a second fraction of hyaluronic acid. The hyaluronic acid of the first fraction may comprise first intermolecular crosslinks. The hyaluronic acid of the second fraction may comprise second intermolecular crosslinks. The composition may comprise one or more additional fractions. The additional fraction(s), when present, may be referred to as “third,” “fourth,” etc. fractions, having “third intermolecular crosslinks,” “fourth intermolecular crosslinks,” etc.

The first fraction may be encapsulated in the second fraction. The second fraction may be encapsulated in the first fraction. For a composition comprising an additional fraction(s), one fraction may encapsulate one or more other fractions. The encapsulating fraction may have a higher crosslink density that fraction(s) encapsulated. The encapsulating fraction may have a lower crosslink density than fraction(s) encapsulated. If placed in an the aqueous medium, the first fraction and/or the second fraction may be in the form of insoluble particles. For a composition comprising an additional fraction(s), additional fraction(s) may be in the form of insoluble particles in aqueous medium.

If placed in an the aqueous medium, the composition may release the hyaluronic acid from the first fraction into a surrounding aqueous medium at a rate different than from the second fraction. For a composition comprising an additional fraction(s), the composition may release the hyaluronic acid from additional fraction(s) a rate different than from the first and/or second fractions.

One or both of the first intermolecular crosslinks and second intermolecular crosslinks may comprise covalent crosslinks. At least a portion, or all, of the covalent crosslinks of one or both of the first intermolecular crosslinks and second intermolecular crosslinks may hydrolytically labile. For a composition comprising an additional fraction(s), at least a portion, or all, of the covalent crosslinks of one or more additional fraction intermolecular crosslinks may be hydrolytically labile. The hydrolytically labile crosslinks may be ester bonds.

The first intermolecular crosslinks may be at a first crosslink density and the second intermolecular crosslinks may be at a second crosslink density. For a composition comprising an additional fraction(s), each respective faction may have respective crosslink density. Crosslink density is a concentration of bonds to HA polymers per unit volume (e.g., moles of crosslink bonds per liter. A link from one HA polymer to another HA polymer would include 2 crosslinking bonds). The selection of crosslink density for each fraction may be independent from other fractions, and the same or different than for other fractions. A crosslink density may be selected from 0.1 mM, 1 mM, 10 mM, 100 mM, 1 M or 10 M, less than any one of 0.1 mM, 1 mM, 10 mM, 100 mM, 1 M or 10 M, greater than one of 0.1 mM, 1 mM, 10 mM, 100 mM, 1 M or 10 M, or in a range between and including any two of 0.1 mM, 1 mM, 10 mM, 100 mM, 1 M or 10 M. The selection may be made such that the first and second densities are in different density groups. The first crosslink density and the second crosslink density may be independently selected from 0.1 mM, 1 mM, 10 mM, 100 mM, 1 M or 10 M, less than any one of 0.1 mM, 1 mM, 10 mM, 100 mM, 1 M or 10 M, greater than one of 0.1 mM, 1 mM, 10 mM, 100 mM, 1 M or 10 M, or in a range between and including any two of 0.1 mM, 1 mM, 10 mM, 100 mM, 1 M or 10 M. Third or higher order crosslink densities may be selected from the same ranges and values.

The first intermolecular crosslinks may be at a first extent of crosslinking and the second intermolecular crosslinks may be at a second extent of crosslinking. For a composition comprising an additional fraction(s), each respective faction may have respective extent of crosslinking. Extent of crosslinking is the percent of repeat units on a polymer that are bound to another polymer or linker molecule through covalent or non-covalent interactions. The selection of an extent of crosslinking for each fraction may be independent from other fractions, and the same or different than for other fractions. An extent of crosslinking may be selected from 0.1, 1, 2, 5, 10, 20, 50, or 100%, less than any one of 0.1, 1, 2, 5, 10, 20, 50, or 100%, greater than any one of 0.1, 1, 2, 5, 10, 20, 50, or 99%, or in a range between and including any two of 0.1, 1, 2, 5, 10, 20, 50, or 100%. The first extent of crosslinking may be different than the second first extent of crosslinking. The first extent of crosslinking may be greater than the second first extent of crosslinking. The first extent of crosslinking may be less than the second first extent of crosslinking. The first extent of crosslinking and the second extent of crosslinking may be independently selected from 0.1, 1, 2, 5, 10, 20, 50, or 100%, less than any one of 0.1, 1, 2, 5, 10, 20, 50, or 100%, greater than any one of 0.1, 1, 2, 5, 10, 20, 50, or 99%, or in a range between and including any two of 0.1, 1, 2, 5, 10, 20, 50, or 100%. Third or additional fraction extents of crosslinking may be selected from the same ranges and values.

As described, a composition herein may comprise one or more additional fraction of hyaluronic acid. The one or more additional fraction of hyaluronic acid may comprise a third fraction of hyaluronic acid. The hyaluronic acid of the third fraction may comprises third intermolecular crosslinks. The third fraction may be encapsulated in the first fraction and/or the second fraction. The encapsulating fraction may have a higher crosslink density that the fraction(s) encapsulated. The encapsulating fraction may have a lower crosslink density than the fraction(s) encapsulated. The third intermolecular crosslinks may be covalent crosslinks. The covalent crosslinks in the third intermolecular crosslinks may be hydrolytically labile. The hydrolytically labile crosslinks may be ester bonds.

The third intermolecular crosslinks may be at a third crosslink density. The third crosslink density may be independently selected compared to the first or second crosslink density. The third crosslink density may be one of 0.1 mM, 1 mM, 10 mM, 100 mM, 1 M or 10 M, less than one of 0.1 mM, 1 mM, 10 mM, 100 mM, 1 M or 10 M, greater than one of 0.1 mM, 1 mM, 10 mM, 100 mM, 1 M or 10 M, or in a range between and including any two of 0.1 mM, 1 mM, 10 mM, 100 mM, 1 M or 10 M.

The third intermolecular crosslinks may be at an extent of crosslinking selected from 0.1, 1, 2, 5, 10, 20, 50, or 100%, less than one of 0.1, 1, 2, 5, 10, 20, 50, or 100%, greater than one of 0.1, 1, 2, 5, 10, 20, 50, or 99%, or in a range between and including any two of 0.1, 1, 2, 5, 10, 20, 50, or 100%.

Intermolecular crosslinks, including the first intermolecular crosslinks, the second intermolecular crosslinks, and/or the third crosslinks, may be bonds formed by an aldehyde, hydrazide, thiol, acrylate, methacrylate, hydroxyethylmethacrylate, norbornene, azide, alkyne, glycidyl methacrylate, haloacetate, benzyl ester, tyramide, glycidyl ether, epoxide, cyclodextrin, adamantane, and ionically charged groups. Intermolecular crosslinks, including the first intermolecular crosslinks, the second intermolecular crosslinks, the third crosslinks, and/or one or more additional fraction intermolecular crosslinks may be hydrolytically degradable. The hydrolytically degradable moieties may be independently selected from the group consisting of lactic acid, poly-L-lactic acid, caprolactone, polycaprolactone, glycolic acid, polyglycolic acid, hydroxyethyl methacrylate, and anhydrides. Intermolecular crosslinks, including the first intermolecular crosslinks, the second intermolecular crosslinks, the third intermolecular crosslinks, and/or any additional fraction intermolecular crosslinks may be degradable by enzymatic catalysis. The enzyme may be a protease or carbohydrate hydrolase.

In an embodiment, the HA is crosslinked with a mechanism known in the art including but not limited to non-covalent, hydrophobic, electrostatic, covalent, free radical, Michael addition, thiol-ene, condensation, etc. In an embodiment, an initiator is added to the HA to initiate crosslinking. In an embodiment, two or more chemically modified HA polymers with complimentary chemical modifications that react upon mixing may be utilized. In an embodiment, two or more chemically modified HA polymers with complimentary chemical modifications that interact upon mixing may be utilized.

A crosslinking molecule may be utilized to form crosslinked HA. Any suitable crosslinking molecule may be utilized. Suitable, in the context of a suitable crosslinking molecule, means it interacts or reacts with two or more HA polymers to form a hydrogel. In an embodiment, the molecule comprises at least one of a peptide, polypeptide, gelatin, hyaluronic acid, dextran, alginate, polyvinylpyrrolidone, methylcellulose, polysaccharides, polycaprolactone, polyvinyl alcohol, or polyethylene glycol. Embodiments include hydrolytically degradable molecules including but not limited to polyesters such as polylactic acid, poly-L-lactic acid, polyglycolic acid, and other hydrolysable chemistries. The crosslinking molecule may have a functionality equal to 2. The crosslinking molecule may have a functionality between 3 and 50,000 or between 2 and 50,000. The functional group of the crosslinking molecule may comprise, consist essentially of, or consist of any of those known in the art including but not limited to at least one of aldehyde, hydrazide, thiol, acrylate, methacrylate, hydroxyethylmethacrylate, norbornene, azide, alkyne, glycidyl methacrylate, haloacetate, benzyl ester, tyramide, glycidyl ether, epoxide, cyclodextrin, adamantane etc. The crosslinking molecule may be a drug.

A composition herein may be in the form of a heterogenous composite with a detectable phase separation or boundaries. The detectable phase separation or boundaries may be between different fractions; e.g., the first fraction and the second fraction, or the first, second, and third fractions.

A constituent of one or more fraction may be a linear hyaluronic acid. The one or more fraction may be the first, the second, the third, or any other fraction. The linear hyaluronic acid may have a molecular weight independently selected for each fraction and greater than 80 KDa, 200 KDa, 500 KDa, 700 KDa, 1000 KDa, 2000 KDa, 3000 KDa, or 4000 KDa, in a range between and including any two of 200 KDa, 500 KDa, 700 KDa, 1000 KDa, 2000 KDa, 3000 KDa, or 4000 KDa, or one of 80 KDa, 200 KDa, 500 KDa, 700 KDa, 1000 KDa, 2000 KDa, 3000 KDa, or 4000 KDa.

The hyaluronic acid in a fraction may chemically modified. The chemical modification may comprise at least one of an aldehyde, hydrazide, thiol, acrylate, methacrylate, hydroxyethylmethacrylate, norbornene, azide, alkyne, glycidyl methacrylate, haloacetate, benzyl ester, tyramide, glycidyl ether, epoxide, cyclodextrin, adamantane, or hydrolytically degradable moieties. The hydrolytically degradable moieties may be selected from one or more of lactic acid, poly-L-lactic acid, caprolactone, polycaprolactone, glycolic acid, polyglycolic acid, hydroxyethyl methacrylate, and anhydrides. The chemical modification may the same or different from one fraction to another.

A composition herein may comprise water or an aqueous buffer. The water or aqueous buffer may surround the hyaluronic acid fractions. The water or aqueous buffer may associate with crosslinked hyaluronic acid networks within the fractions such that the composition is in the form of a hydrogel. The hyaluronic acid concentration in a hydrogel may be greater than 5, 10, 20, 90, 200, 300, 400, 500, 600, 700, 800 or 900 mg/ml, less than 5, 10, 20, 90, 200, 300, 400, 500, 600, 700, 800 or 900 mg/ml, in a range between and including any two of 5, 10, 20, 90, 200, 300, 400, 500, 600, 700, 800 or 900 mg/ml, or one of 5, 10, 20, 90, 200, 300, 400, 500, 600, 700, 800 or 900 mg/ml. The HA hydrogel fractions may have a storage modulus of greater than 10 Pa at 1 Hz. The HA hydrogel fractions may have a storage modulus of greater than 100 Pa at 1 Hz. The storage moduli may be between 200, 1000, 10000 and 50000 Pa at 1 Hz. The HA hydrogel fractions may have a compressive modulus of greater than 100 Pa. The HA hydrogel fractions may have a compressive modulus of greater than 200 Pa at 1 Hz. The compressive moduli may be between 200, 1000, 10000, 50000 and 100000 Pa.

The hyaluronic acid of each fraction may be in particle form. The particle size for each fraction may be independently selected from 1, 10, 100, or 10,000 microns, or in a range between any two of 1, 10, 100, or 10,000 microns, or in a range between any two integers selected from 1 to 10,000 microns.

A composition herein may be in the form of a dry powder. The dry powder may be prepared by precipitating the composition. The dry powder may be prepared by lyophilizing the composition. The composition may comprise one or more excipient. The excipient(s) may stabilize hyaluronic fractions during drying.

A composition herein may comprise a solvent including but not limited polyethylene oxide, polyethylene glycols, and propylene glycol. The composition may comprise an anhydrous solvent. The hyaluronic acid of the composition may be in the solvent or anhydrous solvent.

A composition herein may comprise a drug. The drug may be selected from small molecules, peptides, cytokines, proteins, polysaccharides, synthetic polymers, particles, DNA plasmids, mRNA, cells, cellular exosomes and other cellular components. The drug may be a non-steroidal anti-inflammatory drug. The non-steroidal anti-inflammatory drug is selected from the group consisting of ibuprofen, naproxen, diclofenac, celecoxib, oxaprozin, aspirin, indomethacin, meloxicam, tenoxicam, lornoxicam, piroxicam, ketorolac, sulindac, tolmetin, etodolac, diflunisal, fenoprofen, flurbiprofen, ketoprofen, meclofenamate, nabumetone, aceclofenac, and parecoxib. The drug may be encapsulated in one or more of the fractions. The drug may comprise multiple drugs. Each drug of the multiple drugs may be encapsulated in one or more of the fractions. Sub-combinations of the multiple drugs or each drug of the multiple drugs may be encapsulated in a different fraction. The drug may be separate from the fractions and/or encapsulated in the fractions.

A composition herein may comprise at least one hyaluronidase inhibitor. The hyaluronidase inhibitor may prevent hyaluronidase mediated degradation of one or more of the HA fractions. The at least one hyaluronidase inhibitor may be separate from or embedded in one or more of the fractions. The hyaluronidase inhibitor(s) may be selected from sulfated polysaccharides. The sulfated polysaccharides may be but are not limited to heparin, heparan sulfate, chondroitin sulfate, and synthetically derived molecules. The synthetically derived molecules may be but are not limited to sulfated hyaluronic acid, dextran sulfate, and pentosan polysulfate. The sulfated polysaccharides may be partially or fully sulfated. The partially or fully sulfated polysaccharide described herein can be the pharmaceutically acceptable salt or ester thereof.

A composition herein may comprise one or more antioxidant. The antioxidant(s) may be selected from retinol, alpha tocopherol, ascorbic acid or ascorbic ascorbic acid derivatives including but not limited to ascorbic acid palmitate, L-ascorbic acid 2-glucoside, ascorbyl 3-aminopropyl phosphate, and sodium ascorbyl phosphate.

A composition herein may comprise additional non-hyaluronic acid microparticles or nanoparticles. Additional particles may comprise, consist essentially of, or consist of any of those known in the art including but not limited to poly-L-lactic acid, polyglycolic acid, poly(lactide-co-glycolide), lipids, surfactants, oils, calcium hydroxyapatite, etc. In an embodiment, the additional particles are mixed with the HA fractions. In an embodiment, the additional particles are encapsulated in the HA fractions. Embodiments include particle content in the composition between 0.1, 1, 10, 90% w/w.

A composition herein may comprise a pharmaceutically acceptable carrier. Embodiments include carriers of uncrosslinked or crosslinked HA solutions in aqueous buffer. In an embodiment, the HA carrier concentration is greater than 1 mg/mL. Embodiments include HA carrier concentrations between 10, 20, 40 and 80 mg/mL.

An embodiment comprises a method of treating a subject. The method comprises comprising administering any one or more composition herein to a subject in need thereof. The method may include reconstituting or mixing the one or more composition prior to administration. The administering may be injecting. The administering may be through a catheter. The administering may be with a syringe. The administering may be introducing the composition to a body fluid or tissue of the subject. The administering may be one or more of into synovial fluid of a joint of the subject, into vitreous of an eye of the subject, into an intervertebral spinal disc of the subject, to the vaginal canal of the subject, to skin of the subject, to dermis of the subject, to epidermis of the subject, to a subcutaneous region below the dermis of the subject, or to a pericardial cavity of the subject. The administration may be topical to the skin for hydration. The administration may be to create a non-adhesive barrier between tissues or a tissue and a foreign material.

Any one or more composition herein may be used in a method of treating inflammation. Any one or more composition herein may be used in a method of treating osteoarthritis. Any one or more composition herein may be used in a method of treating ocular disease. Any one or more composition herein may be used in a method of treating cardiovascular disease. Any one or more composition herein may be used in a method of treating disc degeneration. Any one or more composition herein may be used in a method of treating diabetic ulcers. Any one or more composition herein may be used in a method of treating pain. Any one or more composition herein may be used in a method of reducing inflammation following surgery. Any one or more composition herein may be used in a method to improve tissue healing following surgery or a traumatic injury. Any one or more composition herein may be used in a method of treating tissue dryness. Any one or more composition herein may be used in a method to alter skin aesthetics including but not limited to wrinkling filling and increasing tissue hydration and elasticity. Any one or more composition herein may be used in a method to stimulate collagen production. These uses may be accomplished in a method of treating a subject comprising administering the one or more composition to the subject. The administering may comprise injecting the one or more composition to the respective site.

Embodiments include compositions that release hyaluronic acid to tissues or fluids in or on the body. HA release may occur for greater than 1, 2, 4, 10, 30, 90 or 200 days, in a range between and including any two of 1, 2, 4, 10, 30, 90 or 200 days, or any one of 1, 2, 4, 10, 30, 90 or 200 days. Hyaluronic acid mass fraction released per 24 hours may be greater than 0.001, 0.005, 0.01, 0.015, 0.02, 0.033, 0.05, 0.1, 0.14, 0.25 or 0.5, in a range between and including any two of 0.001, 0.005, 0.01, 0.015, 0.02, 0.033, 0.05, 0.1, 0.14, 0.25, 0.5 or 0.9 or any one of 0.001, 0.005, 0.01, 0.015, 0.02, 0.033, 0.05, 0.1, 0.14, 0.25, 0.5 or 0.9.

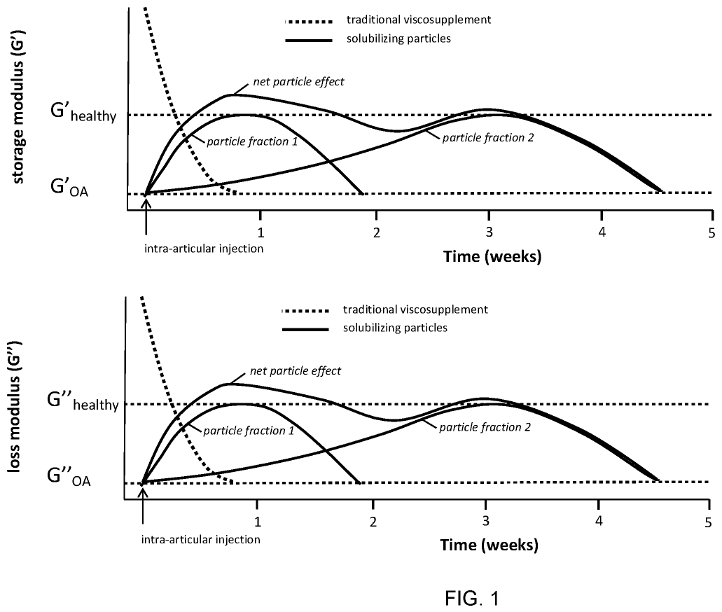

Embodiments include compositions that release hyaluronic acid and increase the viscoelastic properties of tissue and fluids in or on the body. For a non-limiting example see FIG. 1. In embodiments including a method of treating and upon treatment, the viscoelastic properties of the surrounding fluid may increase by a factor of 1.5 or more. The viscoelastic properties of the surrounding fluid may increase by a factor of 2 or more. The viscoelastic properties of the surrounding fluid may increase by a factor of 2.5 or more. Viscoelastic properties include but are not limited to those known in the art including viscosity, zero shear viscosity, complex viscosity, storage modulus, elastic modulus, loss modulus, viscous modulus, etc.

In embodiments including a method of treating and upon treatment, the viscoelastic properties of the surrounding fluid may be increased after 1 hr following administration. The viscoelastic properties of the surrounding fluid may be increased after 24 hrs following administration. The viscoelastic properties of the surrounding fluid may be increased after 2 days following administration. The viscoelastic properties of the surrounding fluid may be increased between 2 and 7 days following administration. Embodiments may include increases in viscoelastic properties for at least or between any two of 2, 7, 30 or 90 days following administration of a composition herein. Embodiments may include increases in viscoelastic properties for at least or between any two of 2, 7, 30, 90, or 180 days following administration of a composition herein.

In embodiments including a method of treating and upon treatment, the storage modulus of the tissue or fluid may increase by 1 Pa, 2 Pa, 5 Pa, or between 5 Pa, or between any two of 10, 50, 100, 200, 500 and 1000 Pa.

In embodiments including a method of treating and upon treatment, the loss modulus of the tissue or fluid may increases by 1 Pa, 2 Pa, 5 Pa, between 5 Pa and 200 Pa, or between any two of 10, 50, 100, 200, 500 and 1000 Pa.

In embodiments including a method of treating and upon treatment, the viscosity of the tissue or fluid may increase by 1 Pa, 2 Pa, 5 Pa, between 5 Pa and 200, between any two of 10, 50, 100, 200, 500 and 1000 Pa. Viscosity values may be determined at shear rates between 0 and 250 1/s including zero shear viscosity values calculated with mathematical models.

Embodiments include compositions that release hyaluronic acid to reduce inflammation. Embodiments include released HA with a molecular weight of greater than 80 KDa, 200 KDa, 500 KDa, 700 KDa, 1000 KDa, 2000 KDa, 3000 KDa, or 4000 KDa, less than 80 KDa, 200 KDa, 500 KDa, 700 KDa, 1000 KDa, 2000 KDa, 3000 KDa, or 4000 KDa, in a range between and including any two of 200 KDa, 500 KDa, 700 KDa, 1000 KDa, 2000 KDa, 3000 KDa, or 4000 KDa, or one of 80 KDa, 200 KDa, 500 KDa, 700 KDa, 1000 KDa, 2000 KDa, 3000 KDa, or 4000 KDa. Embodiments include release HA with an extent of chemical modification or crosslinking less than 100, 50, 25, 15, 10, 5, 2, 1 or 0.5%, in a range between and including any two of 100, 50, 25, 15, 10, 5, 2, 1, or 0.5%, or one of 100, 50, 25, 15, 10, 5, 2, 1 or 0.5%.

Embodiments include compositions that release greater than 0.01, 0.1, 0.2, 0.5, 1, 2, 5, 10, 20, 50, 100 or 200 milligrams of HA per 24 hours, less than 0.01, 0.1, 0.2, 0.5, 1, 2, 5, 10, 20, 50, 100 or 200 milligrams of HA per 24 hours, in a range between and including any two of 0.01, 0.1, 0.2, 0.5, 1, 2, 5, 10, 20, 50, 100 or 200 milligrams of HA per 24 hours, or one of 0.01, 0.1, 0.2, 0.5, 1, 2, 5, 10, 20, 50, 100 or 200 milligrams HA per 24 hours.

A method of treating herein may comprise administering a composition herein to a subject. The HA may be in the form of particles. The particles may be at a concentration greater than 5 mg/mL in aqueous buffer. The particles may be at a concentration greater than 10 mg/mL. The HA particles may be administered at a concentration between 10 mg/mL and 100 mg/mL. Embodiments include HA particle concentrations between any two of 20, 40, 60, 80, 100 and 500 mg/mL, or one of 20, 40, 60, 80, 100 or 500 mg/mL. Embodiments include a dosage form of any composition herein. The dosage form may have HA concentrations between any two of 20, 40, 60, 80, 100 and 500 mg/mL, or one of 20, 40, 60, 80, 100 or 500 mg/mL. Embodiments of the method may comprise administering 0.05. 0.1, 0.5, 1, 2, 3, 4, 5, 6, 7, 8, 9 or 10 mL of composition.

An embodiment comprises a method of making a composition. The method may comprise encapsulating a first dry powder fraction of uncrosslinked HA in a second crosslinked fraction of HA. Embodiments include uncrosslinked HA powders that are processed into microparticles of a uniform size distribution. Embodiments include stabilizing the uncrosslinked HA fractions with salts including divalent cations such as magnesium or calcium. Embodiments include stabilizing the uncrosslinked HA fractions with coatings of polymers, lipids and surfactants. Embodiments include stabilizing the uncrosslinked HA fractions by crosslinking the outer layer of the HA particle. Embodiments include stabilizing the uncrosslinked HA fractions by introducing a layer of crosslinked HA. Embodiments include mixing the uncrosslinked HA fraction into the precursor solution of the second fraction at high concentrations to limit the solubility of the uncrosslinked HA. Embodiments include uncrosslinked HA fraction concentrations of 1, 2, 10, 20, 50, or 80 wt %. Embodiments include dissolving the HA precursor solution of the second fraction at high concentrations to increase viscosity and therefore limit solubility of the first fraction. Embodiments include HA precursor solution concentrations of 1, 2, 5, 10, 20 or 50 wt %. Embodiments include crosslinking the second fraction to limit solubility and release of the encapsulated uncrosslinked HA. Embodiments include processing the crosslinked second fraction with encapsulated first fraction into microparticles with a diameter larger than the diameter of the microparticles of the first fraction. Embodiments include dehydrating the second fraction prior to crosslinking to further increase HA density and crosslink density of the composition.

A method of making herein may comprise encapsulating a first crosslinked HA fraction in a second crosslinked fraction of HA. Embodiments include processing the first fraction into microparticles prior to encapsulation in the second. Embodiments include mixing the first crosslinked HA fraction into the precursor solution of the second fraction at high concentrations to limit the swelling of the first crosslinked fraction. Embodiments include crosslinked HA fraction concentrations of 1, 2, 10, 20, 50, or 80 wt %. Embodiments include dissolving the HA precursor solution of the second fraction at high concentrations to increase viscosity and therefore limit swelling of the first fraction. Embodiments include HA precursor solution concentrations of 1, 2, 5, 10, 20 or 50 wt %. Embodiments include crosslinking the second fraction to limit swelling, crosslink degradation and release of the encapsulated crosslinked HA. Embodiments include processing the crosslinked second fraction with encapsulated first fraction into microparticles with a diameter larger than the diameter of the microparticles of the first fraction. Embodiments include dehydrating the second fraction prior to crosslinking to further increase HA density and crosslink density of the composition.

A method of making herein may comprise encapsulating both uncrosslinked and crosslinked fractions into a third crosslinked fraction. The method may comprise encapsulating two crosslinked fractions into a third fraction. The method may comprise encapsulating two uncrosslinked fractions into a third fraction. Embodiments include processing the encapsulated fractions into microparticles prior to encapsulation in the third. Embodiments include mixing the first and second fractions into the precursor solution of the third fraction at high concentrations to limit the swelling and solubility of the encapsulated fractions. Embodiments include encapsulated HA fraction concentrations of 1, 2, 10, 20, 50, or 80% w/v. Embodiments include dissolving the HA precursor solution of the third fraction at high concentrations to increase viscosity and therefore limit solubility and swelling of the encapsulated fractions. Embodiments include HA precursor solution concentrations of 1, 2, 5, 10, 20 or 50% w/v. Embodiments include crosslinking the third fraction to limit swelling, crosslink degradation and release of the encapsulated HA fractions. Embodiments include processing the crosslinked third fraction with encapsulated fractions into microparticles with a diameter larger than the diameter of the microparticles of the encapsulated fraction. Embodiments include dehydrating the third fraction prior to crosslinking to further increase HA density and crosslink density of the composition.

A method of making herein may comprise making a composition with one or more additional fractions. The one or more additional fractions may be made according to guidelines established above for methods of making involving a first, second, or third fraction.

Embodiments also include the following. Any composition disclosed herein. Any method of making a composition herein. Any composition herein may be used for injection. A method of treatment comprising administering any one or more composition herein to a subject. The subject may be a mammal. The mammal may be, but is not limited, to one selected from humans, horses, and dogs.

Embodiments List. The following list of particular embodiments does limit or exclude embodiments otherwise expressed herein.

1. A composition comprising: a first fraction of hyaluronic acid and a second fraction of hyaluronic acid, wherein when in an aqueous medium the first fraction and the second fraction are insoluble and the composition releases the hyaluronic acid from the first fraction to the aqueous medium at a rate different than from the second fraction. The second fraction may be encapsulated in the first fraction.

2. The composition of embodiment 1, wherein when in aqueous medium the insoluble first fraction and second fraction release hyaluronic acid at an average rate of greater than 0.005, 0.01, 0.05, 0.1 or 0.3 mass fraction of hyaluronic acid per 24 hrs, in a range between and including any two of 0.005, 0.01, 0.05, 0.1 or 0.3 mass fraction of hyaluronic acid per 24 hours, or any one of 0.005, 0.01, 0.05, 0.1 or 0.3 mass fraction of hyaluronic acid per 24 hours, optionally wherein the aqueous medium is at 37° C. and the pH is 7.4.

3. The composition of embodiment 1 or 2, wherein the hyaluronic acid of the first fraction comprises first intermolecular crosslinks, and/or the hyaluronic acid of the second fraction comprises second intermolecular crosslinks.

4. The composition of embodiment 3, wherein one or both of the first intermolecular crosslinks and second intermolecular crosslinks comprise, consist essentially of, or consist of covalent crosslinks.

5. The composition of embodiment 4, wherein at least a portion or all of the covalent crosslinks of one or both of the first intermolecular crosslinks or second intermolecular crosslinks are hydrolytically labile.

6. The composition of embodiment 5, wherein the hydrolytically labile crosslinks comprise, consist essentially of, or consist of ester bonds.

7. The composition of any one or more of embodiments 3-6, wherein the first intermolecular crosslinks are at a first crosslink density and the second intermolecular crosslinks are at a second crosslink density, wherein crosslink density is a concentration of bonds to hyaluronic acid, and optionally the first density is not equal to the second density.

8. The composition of embodiment 7, wherein the first crosslink density and the second crosslink density are independently independently selected from 0.1 mM, 1 mM, 10 mM, 100 mM, 1 M or 10 M, or independently selected from greater than 0.1 mM, 1 mM, 10 mM, 100 mM, 1 M or 10 M, or independently selected from less than 0.1 mM, 1 mM, 10 mM, 100 mM, 1 M or 10 M, or independently in a range between and including any two of 0.1 mM, 1 mM, 10 mM, 100 mM, 1 M or 10 M.

9. The composition of any one or more of embodiments 1-8 further comprising water, wherein the composition is in the form of a hydrogel.

10. The composition of embodiment 9, wherein the hyaluronic acid concentration in the hydrogel is greater than 5, 10, 20, 90, 200, 300, 400, or 500 mg/ml, in a range between and including any two of 5, 10, 20, 90, 200, 300, 400, or 500 mg/ml, or one of 5, 10, 20, 90, 200, 300, 400, or 500 mg/ml.

11. The composition of any one or more of embodiments 3-10, wherein the first intermolecular crosslinks and the second intermolecular crosslinks comprise hydrolytically degradable moieties independently selected from the group consisting of lactic acid, poly-L-lactic acid, caprolactone, polycaprolactone, glycolic acid, polyglycolic acid, hydroxyethyl methacrylate, and anhydrides.

12. The composition of any one or more of embodiments 3-11, wherein one or both of the first intermolecular crosslinks and the second intermolecular crosslinks are degradable by enzymatic catalysis, preferably by a protease or carbohydrate hydrolase.

13. The composition of any one or more of embodiments 1-12, wherein the first fraction and the second fraction form a heterogenous composite with a detectable phase separation or boundary between the first fraction and the second fraction.

14. The composition of any one or more of embodiments 1-13, wherein the hyaluronic acid of each of the first fraction and the second fraction is a linear hyaluronic acid having a molecular weight independently selected for each fraction and greater than 80 KDa, 200 KDa, 500 KDa, 700 KDa, 1000 KDa, 2000 KDa, 3000 KDa, or 4000 KDa, in a range between and including any two of 200 KDa, 500 KDa, 700 KDa, 1000 KDa, 2000 KDa, 3000 KDa, or 4000 KDa, or one of 80 KDa, 200 KDa, 500 KDa, 700 KDa, 1000 KDa, 2000 KDa, 3000 KDa, or 4000 KDa.

15. The composition of any one or more of embodiments 1-14, wherein the hyaluronic acid in at least one of the first fraction or the second fraction is chemically modified, preferably wherein the chemical modification comprises at least one of an aldehyde, hydrazide, thiol, acrylate, methacrylate, hydroxyethylmethacrylate, norbornene, azide, alkyne, glycidyl methacrylate, haloacetate, benzyl ester, tyramide, glycidyl ether, epoxide, cyclodextrin, adamantane, or hydrolytically degradable moieties, preferably where the hydrolytically degradable moieties are selected from one or more of lactic acid, poly-L-lactic acid, caprolactone, polycaprolactone, glycolic acid, polyglycolic acid, hydroxyethyl methacrylate, and anhydrides.

16. The composition of any one or more of embodiments 1-15 further comprising one or more additional fraction of hyaluronic acid.

17. The composition of embodiment 16, wherein the one or more additional fraction of hyaluronic acid comprises a third fraction of hyaluronic acid, the hyaluronic acid of the third fraction comprises third intermolecular crosslinks, and the third fraction is encapsulated in the first fraction.

18. The composition of embodiment 17, wherein the third fraction is encapsulated in the second fraction.

19. The composition of embodiment 17, wherein the one, two, or all three of the first intermolecular crosslinks, the second intermolecular crosslinks, or the third intermolecular crosslinks comprise, consist essentially of, or consist of covalent crosslinks.

20. The composition of embodiment 19, wherein the at least a portion or all of the covalent crosslinks of one, two, or all three of the first intermolecular crosslinks, the second intermolecular crosslinks, or the third intermolecular crosslinks are hydrolytically labile.

21. The composition of embodiment 20, wherein the hydrolytically labile crosslinks comprise, consist essentially of, or consist of ester bonds.

22. The composition of any one or more of embodiments 17-21, wherein the first intermolecular crosslinks are at a first crosslink density, the second intermolecular crosslinks are at a second crosslink density, and the third intermolecular crosslinks are at a third crosslink density, wherein crosslink density is a concentration of bonds to hyaluronic acid, and optionally the first density is not equal to the second density and neither the first density nor the second density are equal to the third density.

23. The composition of any one or more of embodiments 17-22, wherein the first crosslink density, the second crosslink density, and the third crosslink density are

independently selected from 0.1 mM, 1 mM, 10 mM, 100 mM, 1 M or 10 M, or

independently selected from greater than 0.1 mM, 1 mM, 10 mM, 100 mM, 1 M or 10 M, or independently selected from less than 0.1 mM, 1 mM, 10 mM, 100 mM, 1 M or 10 M, or independently in a range between and including any two of 0.1 mM, 1 mM, 10 mM, 100 mM, 1 M or 10 M.

24. The composition of any one or more of embodiments 17-23 further comprising water, wherein the composition is in the form of a hydrogel.

25. The composition of embodiment 24, wherein the hyaluronic acid concentration in the hydrogel is greater than 5, 10, 20, 90, 200, 300, 400, or 500 mg/ml, in a range between and including any two of 5, 10, 20, 90, 200, 300, 400, or 500 mg/ml, or one of 5, 10, 20, 90, 200, 300, 400, or 500 mg/ml.

26. The composition of any one or more of embodiments 17-25, wherein the first intermolecular crosslinks, the second intermolecular crosslinks, and the third intermolecular crosslinks comprise hydrolytically degradable moieties independently selected from the group consisting of lactic acid, poly-L-lactic acid, caprolactone, polycaprolactone, glycolic acid, polyglycolic acid, hydroxyethyl methacrylate, and anhydrides.

27. The composition of any one or more of embodiments 17-26, wherein one, two, or all three of the first intermolecular crosslinks, the second intermolecular crosslinks, and the third intermolecular crosslinks are degradable by enzymatic catalysis, preferably by a protease or carbohydrate hydrolase.

28. The composition of any one or more of embodiments 17-27, wherein the first fraction, the second fraction, and the third fraction form a heterogenous composite and at least two of the first fraction, the second fraction, and the third fraction comprise a detectable phase separation or boundary between said respective fractions.

29. The composition of any one or more of embodiments 17-25, wherein the hyaluronic acid of one or more of the first fraction, the second fraction, and the third fraction is a linear hyaluronic acid having a molecular weight independently selected for each fraction and greater than 80 KDa, 200 KDa, 500 KDa, 700 KDa, 1000 KDa, 2000 KDa, 3000 KDa, or 4000 KDa, in a range between and including any two of 200 KDa, 500 KDa, 700 KDa, 1000 KDa, 2000 KDa, 3000 KDa, or 4000 KDa, or one of 80 KDa, 200 KDa, 500 KDa, 700 KDa, 1000 KDa, 2000 KDa, 3000 KDa, or 4000 KDa.

30. The composition any one or more of embodiments 17-29, wherein the hyaluronic acid in at least one of the first fraction, the second fraction, or the third fraction is chemically modified, preferably wherein the chemical modification comprises at least one of an aldehyde, hydrazide, thiol, acrylate, methacrylate, hydroxyethylmethacrylate, norbornene, azide, alkyne, glycidyl methacrylate, haloacetate, benzyl ester, tyramide, glycidyl ether, epoxide, cyclodextrin, adamantane, or hydrolytically degradable moieties, preferably where the hydrolytically degradable moieties are selected from one or more of lactic acid, poly-L-lactic acid, caprolactone, polycaprolactone, glycolic acid, polyglycolic acid, hydroxyethyl methacrylate, and anhydrides.

31. The composition of any one or more of embodiments 1-30, wherein the hyaluronic acid of one more of the fractions is in particle form with a particle size for each fraction independently selected from 1, 10, 100, or 10,000 microns, or in a range between any two of 1, 10, 100, or 10,000 microns, or in a range between any two integers selected from 1 to 10,000 microns.

32. The composition of any one or more of embodiments 1-8, 11-23, and 26-31, wherein the composition is a dry powder.

33. The composition of any one or more of embodiments 1-32 further comprising at least one drug.

34. The composition of embodiment 33, wherein the at least one drug is selected from small molecules, peptides, cytokines, proteins, polysaccharides, synthetic polymers, particles, DNA plasmids, mRNA, cells, cellular exosomes and other cellular components.

35. The composition of embodiment 33 or 34, wherein the at least one drug comprises a non-steroidal anti-inflammatory drug.

36. The composition of embodiment 35, wherein the non-steroidal anti-inflammatory drug is selected from the group consisting of ibuprofen, naproxen, diclofenac, celecoxib, oxaprozin, aspirin, indomethacin, meloxicam, tenoxicam, lornoxicam, piroxicam, ketorolac, sulindac, tolmetin, etodolac, diflunisal, fenoprofen, flurbiprofen, ketoprofen, meclofenamate, nabumetone, aceclofenac, and parecoxib.

37. The composition of any one or more of embodiments 33-36, wherein the at least one drug is encapsulated in one or more of the fractions.

38. The composition of any one or more of embodiments 1-37 further comprising at least one hyaluronidase inhibitor.

39. The composition of embodiment 38, wherein the at least one hyaluronidase inhibitor is selected from sulfated polysaccharides, heparin, heparan sulfate, chondroitin sulfate, sulfated hyaluronic acid, dextran sulfate and pentosan polysulfate.

40. The composition of any one or more of embodiments 1-39 further comprising an at least one antioxidant, preferably where the at least one antioxidant is selected from the group consisting retinol, alpha tocopherol, ascorbic acid and ascorbic acid derivatives, wherein the ascorbic acid derivatives are selected from the group consisting of ascorbic acid palmitate, L-ascorbic acid 2-glucoside, ascorbyl 3-aminopropyl phosphate, and sodium ascorbyl phosphate.

41. The composition of any one or more of embodiments 1-40 further comprising a pharmaceutically acceptable carrier.

42. A method of treatment comprising administering the composition of any one or more of embodiments 1-41 to a subject in need thereof.

43. The method of embodiment 42, wherein the administering is injecting.

44. The method of embodiment 42 or 43, wherein the administering is introducing the composition to a body fluid or tissue of the subject.

45. The method of any one or more of embodiments 42-44, wherein the administering is into synovial fluid of a joint of the subject, into vitreous of an eye of the subject, into an intervertebral spinal disc of the subject, to skin of the subject, to dermis of the subject, to epidermis of the subject, to a subcutaneous region below the dermis of the subject, into vaginal canal of a subject, or to a pericardial cavity of the subject.

46. A method of making a composition comprising encapsulating a second fraction in a first fraction of hyaluronic acid, wherein the first fraction limits swelling and therefore release of the second fraction, optionally where the composition is the composition of any one of embodiments 1-40.

47. The method of embodiment 46 further comprising encapsulating a second fraction and a third fraction in a first fraction of hyaluronic acid, wherein the first fraction limits swelling and therefore release of the second fraction and third fraction.

48. The method of one of embodiments 46 or 47, wherein the encapsulated fractions are crosslinked at a lower crosslink density than the first fraction prior to being micronized, dehydrated and mixed with the precursor solution of the first fraction, followed by crosslinking of the first fraction and processing the composition into microparticles larger than the encapsulated microparticles.

50. A method of administering hyaluronic acid comprising injecting a composition comprising two insoluble fractions of hyaluronic acid that release hyaluronic acid at different rates into a body through a syringe or catheter, optionally where the composition is the composition of any one of embodiments 1-40. The body may be a subject's body. The body may be the body of a human, a horse, or a dog.

51. The method of embodiment 50, wherein the insoluble fractions of hyaluronic acid release hyaluronic acid at an average rate of greater than 0.005, 0.01, 0.05, 0.1 or 0.3 mass fraction of hyaluronic acid per 24 hrs, in a range between and including any two of 0.005, 0.01, 0.05, 0.1 or 0.3 mass fraction of hyaluronic acid per 24 hours, or any one of 0.005, 0.01, 0.05, 0.1 or 0.3 mass fraction of hyaluronic acid per 24 hours, optionally where the release is at 37° C. and at a pH of 7.4.

52. The method of embodiment 50 or 51, wherein the molecular weight of the released hyaluronic acid is greater than 80 KDa, 200 KDa, 500 KDa, 700 KDa, 1000 KDa, 2000 KDa, 3000 KDa, or 4000 KDa, in a range between and including any two of 200 KDa, 500 KDa, 700 KDa, 1000 KDa, 2000 KDa, 3000 KDa, or 4000 KDa, or one of 80 KDa, 200 KDa, 500 KDa, 700 KDa, 1000 KDa, 2000 KDa, 3000 KDa, or 4000 KDa.

53. The composition of any one or more of embodiments 1-41, wherein the second fraction is encapsulated in the first fraction.

54. A method of treatment comprising administering the composition of embodiment 53 to a subject in need thereof.

55. The method of embodiment 54, wherein the administering is injecting.

56. The method of embodiment 54 or 55, wherein the administering is introducing the composition to a body fluid of the subject.

57. The method of one or more of embodiments 54-56, wherein the administering is into synovial fluid of a joint of the subject, into vitreous of an eye of the subject, into an intervertebral spinal disc of the subject, to skin of the subject, to dermis of the subject, to epidermis of the subject, to a subcutaneous region below the dermis of the subject, into vaginal canal of a subject, or to a pericardial cavity of the subject.

### EXAMPLES

The following examples represent particular non-limiting embodiments. Any embodiment herein may be supplemented with one or more detail from an example herein.

### Example 1

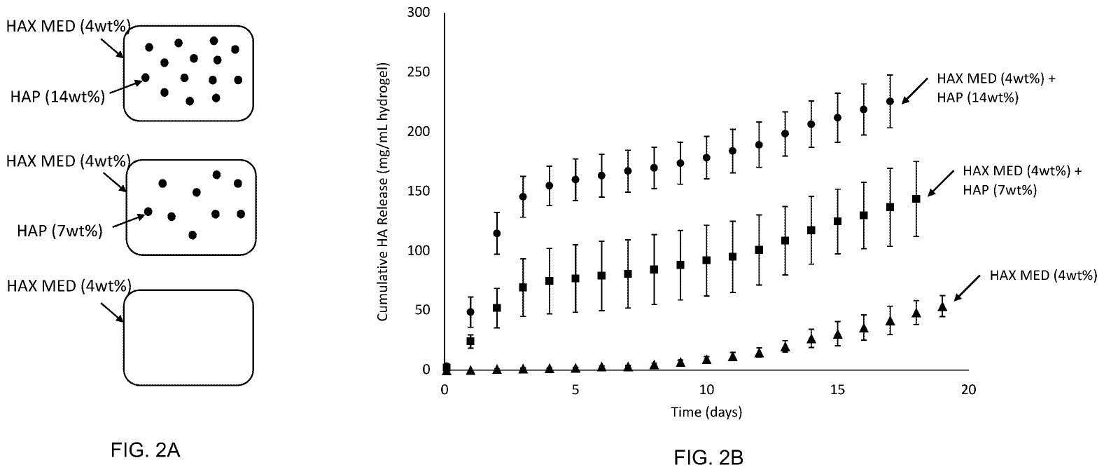

Sodium hyaluronate (1.5 MDa, LifeCore Biomedical) was precipitated in ethanol, dried, milled and sieved to yield particles with diameters between 25 and 53 microns (HAP). Sodium hyaluronate (700 kDa, LifeCore Biomedical) was chemically modified with hydroxyethyl methacrylate groups through an esterification reaction, purified and characterized with 1H NMR to determine a 11% extent of modification. The modified HA was dissolved at 40 mg/mL (10.4 mM methacrylate groups) in aqueous pH 8.5 buffer, the HA particles were added to the precursor solution at 0, 35 or 70 mg/mL, and dithiothreitol was added at a 1:1 thiol:methacrylate ratio and incubated overnight at 37° C. to form HA hydrogels with one or two fractions (FIG. 2A). The two-phase HA hydrogels were incubated in PBS at 37° C. and 60 rotations per minute. The buffer was collected every day and replaced with fresh buffer and HA content in the buffer was quantified with a carbazole based uronic acid assay. Cumulative HA release per milliliter of the starting hydrogel is presented in FIG. 2B.

### Example 2

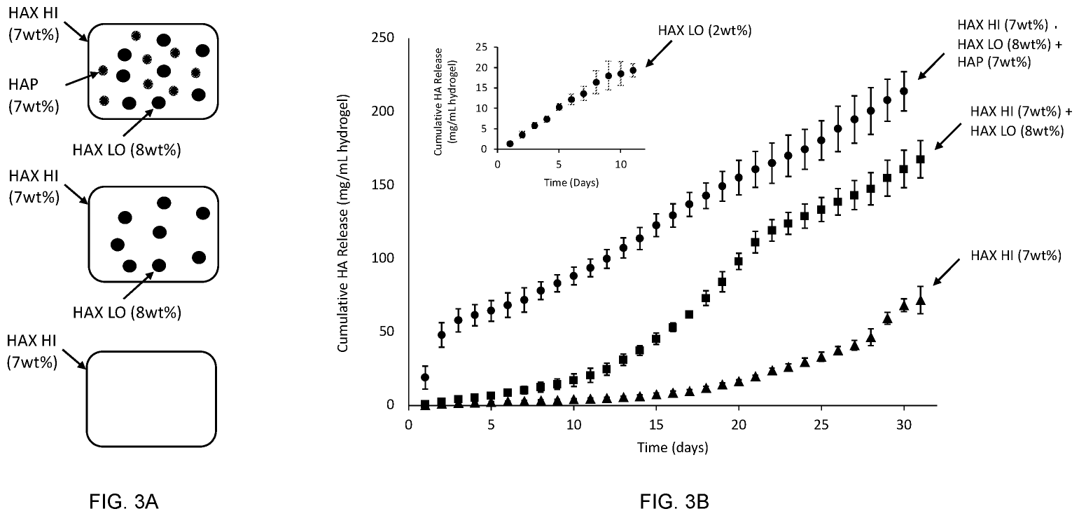

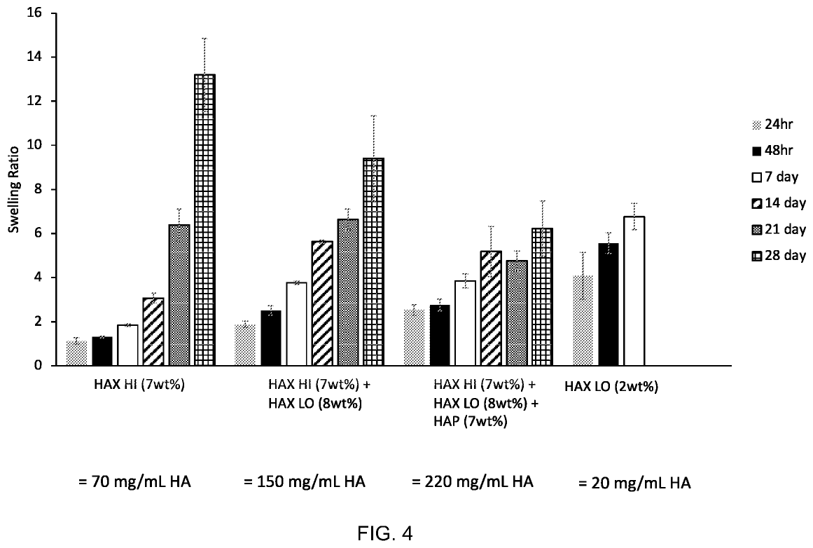

Sodium hyaluronate (1.5 MDa, LifeCore Biomedical) was precipitated in ethanol, dried, milled and sieved to yield particles with diameters between 25 and 53 microns (HAP). Sodium hyaluronate (700 kDa, LifeCore Biomedical) was chemically modified with hydroxyethyl methacrylate groups through an esterification reaction, purified and characterized with 1H NMR to determine a 7% extent of modification. The modified HA was dissolved at 40 mg/mL (6.7 mM methacrylate groups, HAX LO) in aqueous pH 8.5 buffer, dithiothreitol was added at a 1:1 thiol:methacrylate ratio, incubated overnight at 37° C., fragmented into microparticles, dehydrated in ethanol, and dried to form a powder. Sodium hyaluronate (700 kDa, LifeCore Biomedical) was chemically modified with hydroxyethyl methacrylate groups through an esterification reaction, purified and characterized with 1H NMR to determine a 16% extent of modification. The modified HA was dissolved at 70 mg/mL (22 mM methacrylate groups, HAX HI) in aqueous pH 8.5 buffer, HAP and HAX LO particles were added to the precursor solution at 0, 70 or 80 mg/mL, dithiothreitol was added at a 1:1 thiol:methacrylate ratio and incubated overnight at 37° C. to form two and three fraction hydrogels (FIG. 3A). The HA hydrogels were incubated in PBS at 37° C. and 60 rotations per minute. The buffer was collected every day and replaced with fresh buffer and HA content in the buffer was quantified with a carbazole based uronic acid assay. Cumulative HA release per milliliter of the starting hydrogel is presented in (FIG. 3B). The mass of the hydrogels were determined after 24 hours, 48 hours, 7 days, 14 days, 21 days and 28 days. Swelling ratios were determined by taking the increase in the hydrogel mass from the pre-swelled mass and dividing by the pre-swelled mass (FIG. 4).

### Example 3

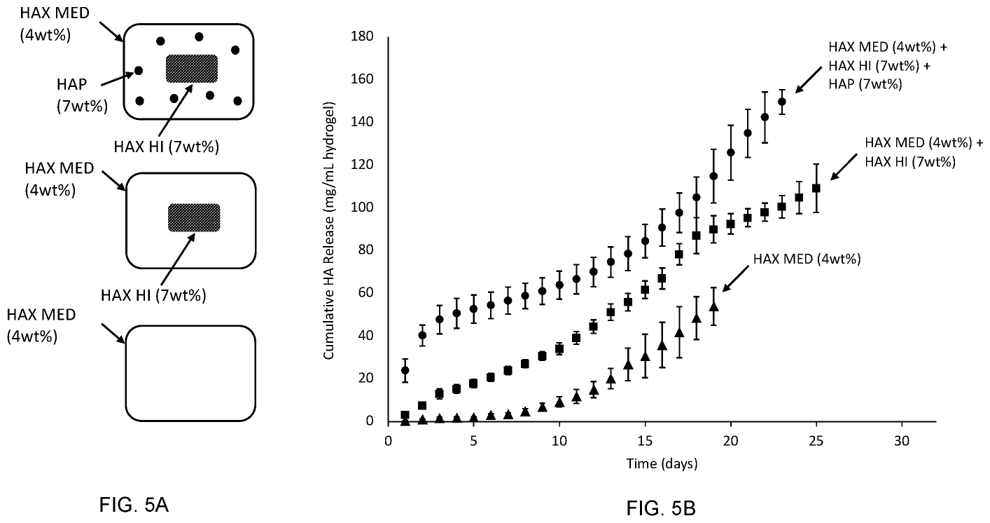

Sodium hyaluronate (1.5 MDa, LifeCore Biomedical) was precipitated in ethanol, dried, milled and sieved to yield particles with diameters between 25 and 53 microns (HAP). Sodium hyaluronate (700 kDa, LifeCore Biomedical) was chemically modified with hydroxyethyl methacrylate groups through an esterification reaction, purified and characterized with 1H NMR to determine a 16% extent of modification. The modified HA was dissolved at 70 mg/mL (22 mM methacrylate groups, HAX HI) in aqueous pH 8.5 buffer, dithiothreitol was added at a 1:1 thiol:methacrylate ratio, incubated overnight at 37° C., dehydrated in ethanol, and dried. Sodium hyaluronate (700 kDa, LifeCore Biomedical) was chemically modified with hydroxyethyl methacrylate groups through an esterification reaction, purified and characterized with 1H NMR to determine a 11% extent of modification. The modified HA was dissolved at 40 mg/mL (11 mM methacrylate groups, HAX MED) in aqueous pH 8.5 buffer and HAPs and dehydrated HAX HI gels were added to the precursor solution at 0 or 70 mg/mL, dithiothreitol was added at a 1:1 thiol:methacrylate ratio and incubated overnight at 37° C. to form two or three phase hydrogels (FIG. 5A). The HA hydrogels were incubated in PBS at 37° C. and 60 rotations per minute. The buffer was collected every day and replaced with fresh buffer and HA content in the buffer was quantified with a carbazole based uronic acid assay. Samples were collected until the HA hydrogels could no longer be separated from the solution. Cumulative HA release per milliliter of the starting hydrogel is presented in (FIG. 5B).

### Example 4

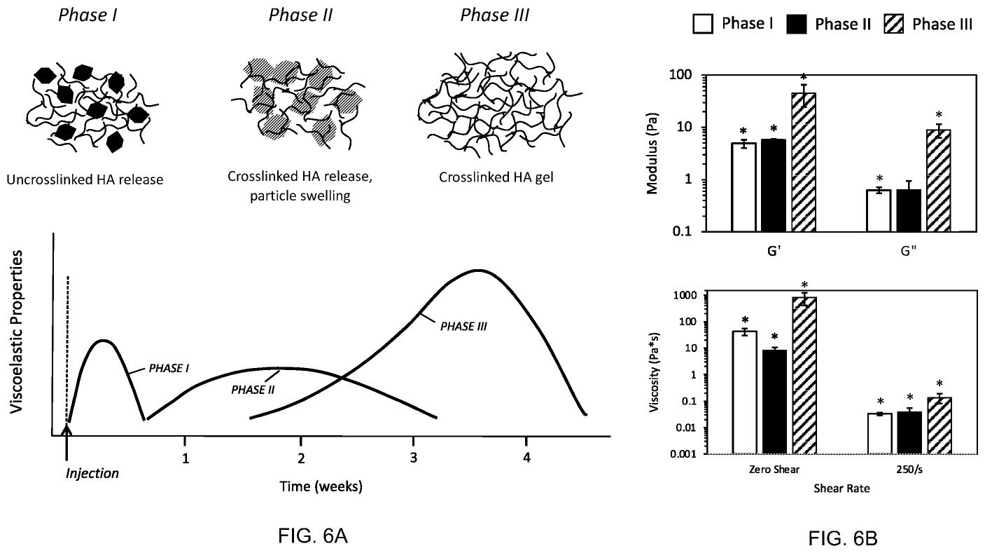

HA hydrogels were fabricated with three fractions as in Example 2: 7 wt % HAX HI, 8 wt % HAX LO, 7 wt % HAP. These gels were fragmented into microparticles by extruding through a 120 micron opening and incubated in PBS at a concentration of 50 microliter gel to 300 microliter buffer and incubated at 37° C. and 60 rotations per minute. Supernatant buffer was collected every day and replaced with fresh buffer. The viscoelastic properties of the materials were measured after 2, 15 and 30 days with rheology using a 20 mm plate geometry on a TA HR20 rheometer (FIG. 6). Storage and loss modulus were measured with 0.5% strain and 2.5 Hz. Viscosity values were measured over 1E-3 to 1E3 1/s shear rates. All measurements were performed at 22° C.

### Example 5

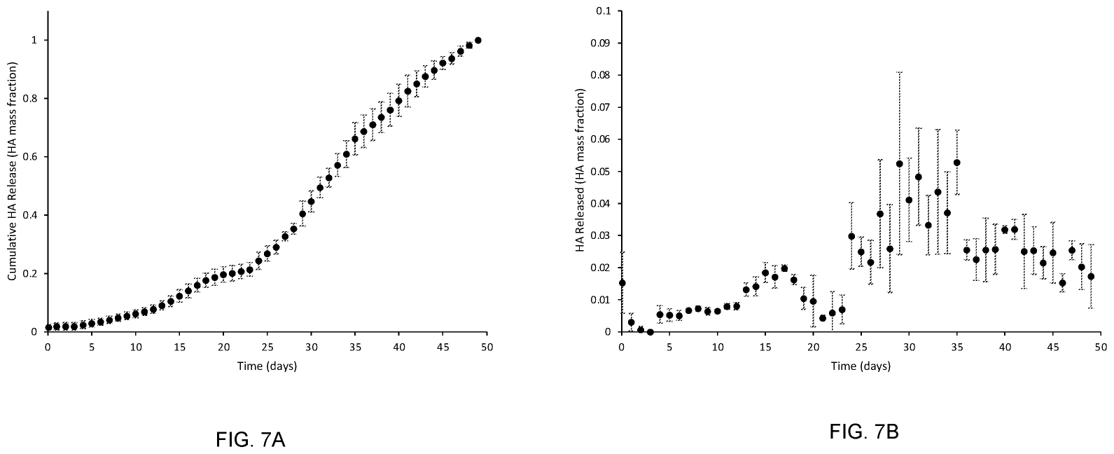

Sodium hyaluronate (1.5 MDa, LifeCore Biomedical) was precipitated in ethanol, dried, milled and sieved to yield particles with diameters between 25 and 53 microns. Sodium hyaluronate (700 kDa, LifeCore Biomedical) was chemically modified with hydroxyethyl methacrylate groups through an esterification reaction, purified and characterized with 1H NMR to determine an 8.5% extent of modification. The modified HA was dissolved at 30 mg/mL in aqueous pH 8.5 buffer (6.1 mM methacrylate groups), dithiothreitol was added at a 1:1 thiol:methacrylate ratio, incubated overnight at 37° C., fragmented into microparticles through a 58 micron mesh, washed for 1 hr in water, dehydrated in ethanol, and dried to form a powder. Sodium hyaluronate (700 kDa, LifeCore Biomedical) was chemically modified with hydroxyethyl methacrylate groups through an esterification reaction, purified and characterized to determine a 20% extent of modification. The modified HA was dissolved at 50 mg/mL (23 mM methacrylate groups) in aqueous pH 8.5 buffer and the uncrosslinked and crosslinked HA particles were added to the precursor solution at 40 mg/mL and 110 mg/mL, respectively, dithiothreitol was added at a 1:1 thiol:methacrylate ratio, and incubated overnight at 37° C. The hydrogels were fragmented into microparticles through a 180 micron mesh, washed for 1 hr in water, dehydrated in ethanol and dried to a powder. 8 mg of powder was incubated in 1.5 mL of phosphate buffered saline and incubated in PBS at 37° C. and 80 rotations per minute. The particles were separated from the supernatant using an 8 micron polycarbonate mesh and supernatant buffer was collected every day and replaced with fresh buffer and HA content in the buffer was quantified with a carbazole based uronic acid assay and presented as a mass fraction of total HA (FIGS. 7A and 7B).

### Example 6

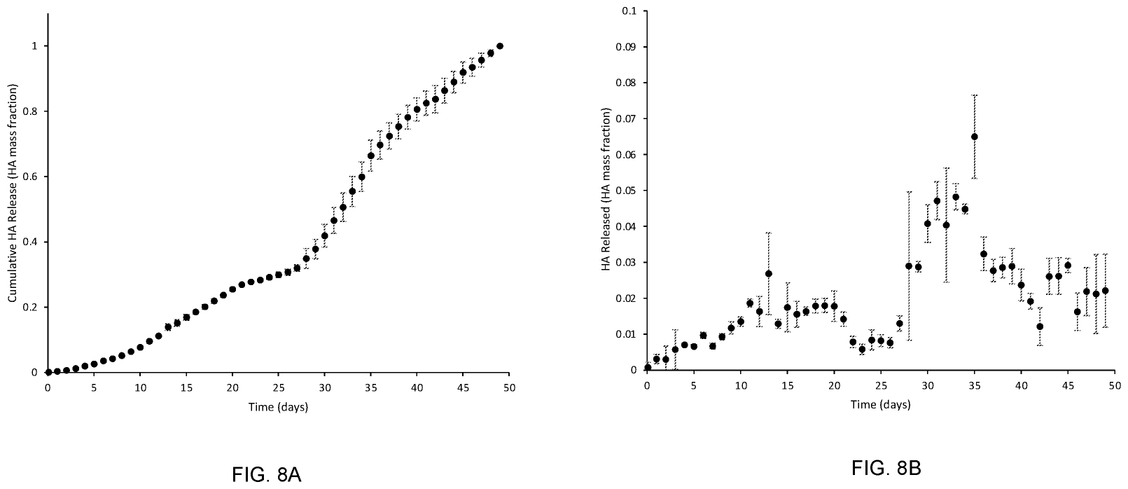

Sodium hyaluronate (700 kDa, LifeCore Biomedical) was chemically modified with hydroxyethyl methacrylate groups through an esterification reaction, purified and characterized with 1H NMR to determine a 5.6% extent of modification. The modified HA was dissolved at 20 mg/mL (2.7 mM methacrylate groups) in aqueous pH 8.5 buffer, dithiothreitol was added at a 1:1 thiol:methacrylate ratio, incubated overnight at 37° C., fragmented into microparticles through a 58 micron screen, washed for 1 hr in water, dehydrated in ethanol, and dried to form a powder. Sodium hyaluronate (700 kDa, LifeCore Biomedical) was chemically modified with hydroxyethyl methacrylate groups through an esterification reaction, purified and characterized with 1H NMR to determine a 11.2% extent of modification. The modified HA was dissolved at 30 mg/mL (7.9 mM methacrylate groups) in aqueous pH 8.5 buffer, dithiothreitol was added at a 1:1 thiol:methacrylate ratio, incubated overnight at 37° C., fragmented into microparticles through a 58 micron screen, washed for 1 hr in water, dehydrated in ethanol, and dried to form a powder. Sodium hyaluronate (700 kDa, LifeCore Biomedical) was chemically modified with hydroxyethyl methacrylate groups through an esterification reaction, purified and characterized with 1H NMR to determine a 20% extent of modification. The modified HA was dissolved at 60 mg/mL (27 mM methacrylate groups) in aqueous pH 8.5 buffer and the crosslinked HA particles were added to the precursor solution at 85 mg/mL and 55 mg/mL, dithiothreitol was added at a 1:1 thiol:methacrylate ratio and incubated overnight at 37° C. The hydrogels were fragmented into microparticles through a 180 micron screen, washed for 1 hr in water, dehydrated in ethanol and dried to a powder. 8 mg of powder was incubated in 1.5 mL of phosphate buffered saline and incubated in PBS at 37° C. and 80 rotations per minute. The particles were separated from the supernatant using an 8 micron polycarbonate mesh and supernatant buffer was collected every day and replaced with fresh buffer and HA content in the buffer was quantified with a carbazole based uronic acid assay and presented as a mass fraction of total HA (FIGS. 8A and 8B).

### Example 7

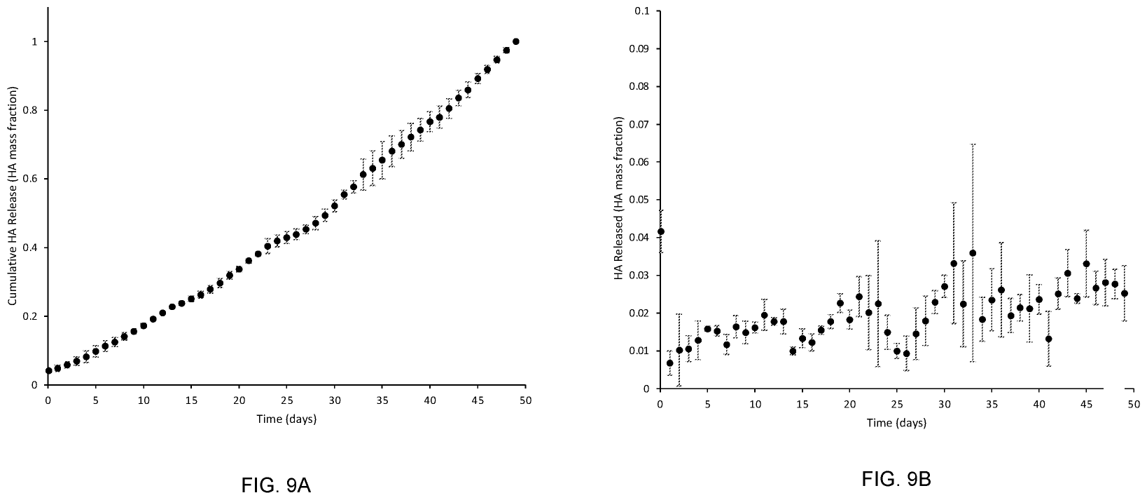

Sodium hyaluronate (1.5 MDa, LifeCore Biomedical) was precipitated in ethanol, dried, milled and sieved to yield particles with diameters between 25 and 53 microns. Sodium hyaluronate (700 kDa, LifeCore Biomedical) was chemically modified with a hydroxyethyl methacrylate groups through an esterification reaction, purified and characterized with 1H NMR to determine a 5.6% extent of modification. The modified HA was dissolved at 30 mg/mL (4.1 mM methacrylate groups) in aqueous pH 8.5 buffer, dithiothreitol was added at a 1:1 thiol:methacrylate ratio, incubated overnight at 37° C., fragmented into microparticles through a 58 micron screen, washed for 1 hr in water, dehydrated in ethanol, and dried to form a powder. Sodium hyaluronate (700 kDa, LifeCore Biomedical) was chemically modified with hydroxyethyl methacrylate groups through an esterification reaction, purified and characterized with 1H NMR to determine a 13.7% extent of modification. The modified HA was dissolved at 50 mg/mL (16 mM methacrylate groups) in aqueous buffer and the uncrosslinked and crosslinked HA particles were added to the precursor solution at 50 mg/mL and 100 mg/mL, respectively and dithiothreitol was added at a 1:1 thiol:methacrylate ratio and incubated overnight at 37° C. The hydrogels were fragmented into microparticles through a 180 micron screen, washed for 1 hr in water, dehydrated in ethanol and dried to a powder. 8 mg of powder was incubated in 1.5 mL of phosphate buffered saline and incubated in PBS at 37° C. and 80 rotations per minute. The particles were separated from the supernatant using an 8 micron polycarbonate mesh and supernatant buffer was collected every day and replaced with fresh buffer and HA content in the buffer was quantified with a carbazole based uronic acid assay and presented as a mass fraction of total HA (FIGS. 9A and 9B).

### Example 8

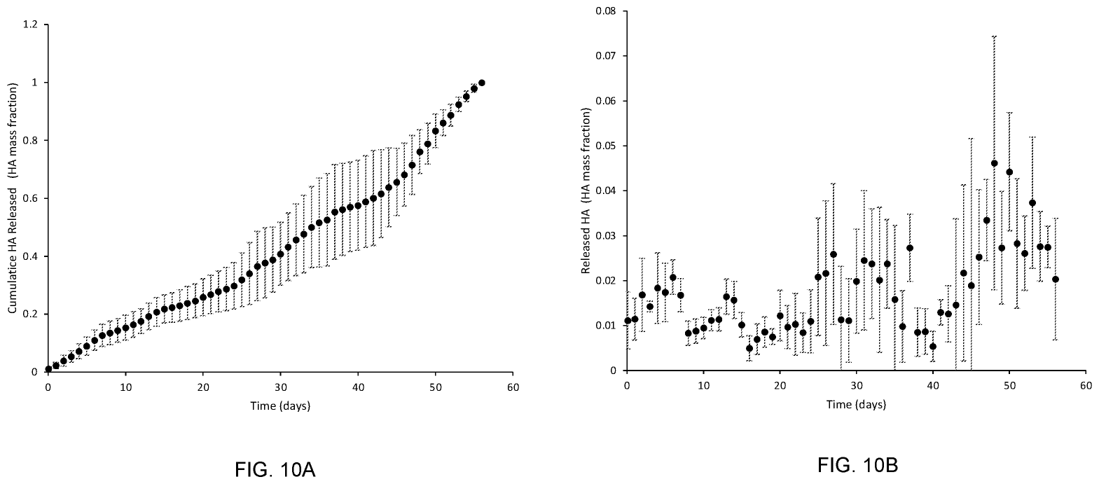

Sodium hyaluronate (1.5 MDa, LifeCore Biomedical) was precipitated in ethanol, dried, milled and sieved to yield particles with diameters between 25 and 53 microns. Sodium hyaluronate (700 kDa, LifeCore Biomedical) was chemically modified with hydroxyethyl methacrylate groups through an esterification reaction, purified and characterized with 1H NMR to determine a 6.9% extent of modification. The modified HA was dissolved at 20 mg/mL (3.3 mM methacrylate groups) in aqueous pH 8.5 buffer, dithiothreitol was added at a 1:1 thiol:methacrylate ratio, incubated overnight at 37° C., fragmented into microparticles through a 58 micron screen, washed for 1 hr in water, dehydrated in ethanol, and dried to form a powder. Sodium hyaluronate (700 kDa, LifeCore Biomedical) was chemically modified with hydroxyethyl methacrylate groups through an esterification reaction, purified and characterized with 1H NMR to determine a 22% extent of modification. The modified HA was dissolved at 20 mg/mL (9.9 mM methacrylate groups) in aqueous pH 8.5 buffer, dithiothreitol was added at a 1:1 thiol:methacrylate ratio, incubated overnight at 37° C., fragmented into microparticles through a 58 micron screen, washed for 1 hr in water, dehydrated in ethanol, and dried to form a powder. Sodium hyaluronate (700 kDa, LifeCore Biomedical) was chemically modified with hydroxyethyl methacrylate groups through an esterification reaction, purified and characterized with 1H NMR to determine a 26% extent of modification. The modified HA was dissolved at 50 mg/mL (28 mM methacrylate groups) in aqueous pH 8.5 buffer and the uncrosslinked, crosslinked and higher crosslinked HA particles were added to the precursor solution at 40 mg/mL, 40 mg/mL and 70 mg/mL, respectively and dithiothreitol was added at a 1:1 thiol:methacrylate ratio and incubated overnight at 37° C. The hydrogels were fragmented into microparticles through a 180 micron screen, washed for 1 hr in water, dehydrated in ethanol and dried to a powder. 8 mg of powder was incubated in 1.5 mL of phosphate buffered saline and incubated in PBS at 37° C. and 80 rotations per minute. The particles were separated from the supernatant using an 8 micron polycarbonate mesh and supernatant buffer was collected every day and replaced with fresh buffer and HA content in the buffer was quantified with a carbazole based uronic acid assay and presented as a mass fraction of total HA (FIGS. 10A and 10B).

### Example 9

The three-fraction microparticles fabricated in Example 7 were mixed with poly-L-lactic acid microparticles as dry powders. The microparticles powders were then reconstituted with a solution of 0.5% w/v 1.5 MDa sodium hyaluronate at final concentrations of 2% w/v three-fraction HA microparticles and 1% w/v PLA microparticles. The rheological properties of the composition were G′@0.5 Hz=851 Pa, G″ @0.5 Hz=289 Pa, G′@ 2.5 Hz=1128, G″ @ 2.5 Hz=276 Pa, zero shear viscosity=8438 Pa, viscosity @ 100/s shear rate=3.3 Pa. 100 microliters of the composition was injected subcutaneously into a rat and left a surface feature that remained for at least 7 days. For comparison, 100 microliters of Sculptra (poly-L-lactic acid with carboxymethylcellulose carrier) was injected subcutaneously and left a surface feature which was gone 24 hours after the injection.

The skilled artisan would readily understand that a composition herein could be likewise reconstituted, if starting as a dry powder, and/or injected subcutaneously to sculpt surface features. Compositions with longer or shorter release rates could be utilized to achieve longer or shorter persistence of surface features. When combined with collagen stimulating materials, the surface features could be replaced with collagen as the HA is released. Embodiments comprise a composition herein further comprising collagen. Embodiments comprise methods of treating comprising administering a composition herein that further comprises collagen.

### Example 10

The three-fraction microparticles fabricated in Example 7 could be rehydrated with 0.5% w/v 1.5 MDa sodium hyaluronate at a final concentration of 2% w/v three-fraction HA microparticles. 3 mL of the resulting viscous gel could then be loaded into a single use applicator syringe and injected into the vaginal canal to treat vaginal dryness.

The skilled artisan would understand that compositions herein could be likewise rehydrated, if starting as a dry material, and/or loaded into a single use applicator syringe for injection into the vaginal canal to treat vaginal dryness.

One or more embodiments herein may be combined with one or more other embodiments herein to create additional embodiments. An embodiment disclosed herein is not exclusive of combination with one or more other embodiments herein unless by its terms it excludes elements of the other embodiments.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

### REFERENCES

Snetkov, P., et al., Hyaluronic Acid: The Influence of Molecular Weight on Structural, Physical, Physico-Chemical, and Degradable Properties of Biopolymer. Polymers (Basel), 2020. 12(8).
Balazs, E. A., The Physical Properties of the Synovial Fluid and the Special Role of Hyaluronic Acid, in Disorders of the Knee, A. Helfet, Editor. 1974, T. B. Lippincott Company: Philadelphia. p. 63-75.
Mazzucco, D., R. Scott, and M. Spector, Composition of joint fluid in patients undergoing total knee replacement and revision arthroplasty: correlation with flow properties. Biomaterials, 2004. 25(18): p. 4433-45.
Fam, H., J. T. Bryant, and M. Kontopoulou, Rheological properties of synovial fluids. Biorheology, 2007. 44(2): p. 59-74.
Bhuanantanondh, P., Rheological Study of Viscosupplements and Synovial Fluid in Patients with Osteoarthritis. Journal of Medical and Biological Engineering, 2012. 32: p. 12-16.
Petrey, A. C. and C. A. de la Motte, Hyaluronan, a crucial regulator of inflammation. Front Immunol, 2014. 5: p. 101.
Tram, N. K. and K. E. Swindle-Reilly, Rheological Properties and Age-Related Changes of the Human Vitreous Humor. Front Bioeng Biotechnol, 2018. 6: p. 199.
Quero, L., et al., Hyaluronic acid fragments enhance the inflammatory and catabolic response in human intervertebral disc cells through modulation of toll-like receptor 2 signalling pathways. Arthritis Res Ther, 2013. 15(4): p. R94.
Smith, M. M. and P. Ghosh, The synthesis of hyaluronic acid by human synovial fibroblasts is influenced by the nature of the hyaluronate in the extracellular environment. Rheumatol Int, 1987. 7(3): p. 113-22.
Maneiro, E., et al., The biological action of hyaluronan on human osteoarthritic articular chondrocytes: the importance of molecular weight. Clin Exp Rheumatol, 2004. 22(3): p. 307-12.
Levick, J. R. and J. N. McDonald, Fluid movement across synovium in healthy joints: role of synovial fluid macromolecules. Ann Rheum Dis, 1995. 54(5): p. 417-23.
Brown, T. J., U. B. Laurent, and J. R. Fraser, Turnover of hyaluronan in synovial joints: elimination of labelled hyaluronan from the knee joint of the rabbit. Exp Physiol, 1991. 76(1): p. 125-34
Wobig, M., et al., The role of elastoviscosity in the efficacy of viscosupplementation for osteoarthritis of the knee: a comparison of hylan G-F 20 and a lower-molecular-weight hyaluronan. Clin Ther, 1999. 21(9): p. 1549-62.
Agerup, B., P. Berg, and C. Akermark, Non-animal stabilized hyaluronic acid: a new formulation for the treatment of osteoarthritis. BioDrugs, 2005. 19(1): p. 23-30.
Nicholls, M., et al., Rheological Properties of Commercially Available Hyaluronic Acid Products in the United States for the Treatment of Osteoarthritis Knee Pain. Clin Med Insights Arthritis Musculoskelet Disord, 2018. 11: p. 1179544117751622.
La Gatta, A., et al., Hyaluronan-based hydrogels as dermal fillers: The biophysical properties that translate into a “volumetric” effect. PLoS One, 2019. 14(6): p. e0218287.
Larsen, N. E., et al., Clearance kinetics of a hylan-based viscosupplement after intra-articular and intravenous administration in animal models. J Biomed Mater Res B Appl Biomater, 2012. 100(2): p. 457-62.
Jackson, D. W. and T. M. Simon, Intra-articular distribution and residence time of Hylan A and B: a study in the goat knee. Osteoarthritis Cartilage, 2006. 14(12): p. 1248-57.
De Boulle, K., et al., A review of the metabolism of 1,4-butanediol diglycidyl ether-crosslinked hyaluronic acid dermal fillers. Dermatol Surg, 2013. 39(12): p. 1758-66.
Meyer, K., The biological significance of hyaluronic acid and hyaluronidase. Physiol Rev, 1947. 27(3): p. 335-59.
Mathews, M. B. and A. Dorfman, Inhibition of hyaluronidase. Physiol Rev, 1955. 35(2): p. 381-402.
Feng, Q., et al., Sulfated hyaluronic acid hydrogels with retarded degradation and enhanced growth factor retention promote hMSC chondrogenesis and articular cartilage integrity with reduced hypertrophy. Acta Biomater, 2017. 53: p. 329-342.
Burdick, J. A. and G. D. Prestwich, Hyaluronic acid hydrogels for biomedical applications. Adv Mater, 2011. 23(12): p. H41-56.

It is understood, therefore, that this invention is not limited to the particular embodiments disclosed, but is intended to cover all modifications which are within the spirit and scope of the invention as defined by the appended claims; the above description; and/or shown in the attached drawings.

1. A composition comprising: a first fraction of hyaluronic acid and a second fraction of hyaluronic acid, wherein when in an aqueous medium
wherein the second fraction is encapsulated in the first fraction,
the hyaluronic acid of the first fraction or the second fraction comprises intermolecular crosslinks, and the intermolecular crosslinks are hydrolytically labile.

2.-13. (canceled)

14. The composition of claim 1, wherein the hyaluronic acid of each of the first fraction and the second fraction is a linear hyaluronic acid having a molecular weight greater than 200 KDa.

15. The composition of claim 1, wherein the hyaluronic acid in at least one of the first fraction or the second fraction is chemically modified.

16. The composition of claim 1 further comprising one or more additional fraction of hyaluronic acid.

17.-37. (canceled)

38. The composition of claim 1 further comprising at least one hyaluronidase inhibitor.

39. The composition of claim 38, wherein the at least one hyaluronidase inhibitor is selected from sulfated polysaccharides, heparin, heparan sulfate, chondroitin sulfate, sulfated hyaluronic acid, dextran sulfate and pentosan polysulfate.

40. (canceled)

41. (canceled)

42. (canceled)

43. A method of treatment comprising administering the composition of claim 1 to a subject in need thereof.

44. The method of claim 43, wherein the administering is injecting.

45. The method of claim 43, wherein the administering is introducing the composition to a body fluid or tissue of the subject.

46. The method of claim 43, wherein the administering is into synovial fluid of a joint of the subject, into vitreous of an eye of the subject, into an intervertebral spinal disc of the subject, to skin of the subject, to dermis of the subject, to epidermis of the subject, to a subcutaneous region below the dermis of the subject, into vaginal canal of a subject, or to a pericardial cavity of the subject.

47. A method of making a composition comprising encapsulating a second fraction in a first fraction of hyaluronic acid, wherein the first fraction limits swelling and therefore release of the second fraction.

48. The method of claim 47 further comprising encapsulating a second fraction and a third fraction in a first fraction of hyaluronic acid, wherein the first fraction limits swelling and therefore release of the second fraction and third fraction.

49. (canceled)

50. A method of administering hyaluronic acid comprising injecting a composition comprising a first fraction of hyaluronic acid crosslinked with hydrolytically liable crosslinks, and a second fraction of hyaluronic acid, where the first fraction and the second fraction release hyaluronic acid at different rates into a body through a syringe or catheter.

51.-57. (canceled)

58. The composition of claim 1, wherein when placed in a 20 fold excess of the aqueous media under physiological conditions, the hyaluronic acid in the second fraction becomes uniformly dissolved into solution at a rate different than the rate that the hyaluronic acid in the first fraction

59. The composition of claim 58, wherein the second fraction of hyaluronic acid becomes uniformly dissolved at least 7 days faster than the first fraction

60. The composition of claim 58, wherein the second fraction of hyaluronic acid becomes uniformly dissolved at least 7 days slower than the first fraction

61. The composition of claim 1, wherein when placed in a 20 fold excess of the aqueous media under physiological conditions, the hyaluronic acid in the first fraction and the hyaluronic acid in the second fraction becomes uniformly dissolved in less than 180 days.

62. The composition of claim 61, wherein the hyaluronic acid of each of the first fraction and the second fraction when fully dissolved into solution is a linear hyaluronic acid having a molecular weight greater than 200 KDa.

63. The composition of claim 62, wherein the molecular weight is greater than 500 KDa.

64. The composition of claim 62, wherein the molecular weight is in a range between and including 500 KDa and 2000 KDa.

65. The composition of claim 1, wherein the hyaluronic acid in the first fraction comprises hyaluronic acid particles with a diameter between 1 and 10,000 microns.

66. The composition of claim 1, wherein the hydrolytically degradable crosslinks are ester bonds.

67. The composition of claim 1, wherein hyaluronic acid of the second fraction comprises second intermolecular crosslinks, and the second intermolecular crosslinks are hydrolytically labile.

68. The composition of claim 64, wherein the hyaluronic acid of the second fraction comprises hyaluronic acid particles with diameters between 1 and 10,000 microns.

69. The composition of claim 14, wherein the molecular weight is greater than 500 KDa.

70. The composition of claim 14, wherein the molecular weight is in a range between and including 500 KDa and 2000 KDa.
